SEQUENCE LISTING (12 sequences)

SEQ ID NO: 1 - DNA, 1476 bp, Glycine max
atggaatcagcggcaagaacaacaacaacaacaactcatatagcccttgtttctatcccagctttcagccaccaagtctcaatcctcgagttcgcaaaacgtctccttaatctccacaacaacaccttcaacatcacatgcatcattccaacacttaactcctcttacaataacattgccaccaaacccttctttgattccctccctccgaacattcactgcattttcctcccctcagtgtacttcgaggacctaaacaacaatggagtctctgtggagatccaaatccagctctcggtttctcgcgccatgccctccgttagggaaaccctaagatcactctttgatgccaccaacaacgtcgtcgccatcgttgccgatgccatggtcccggaggcgctcgattttggtaaggaactcggcatcttatcctacatctacttcccttgttccacaatgttgctatccctatgccttcattcttcaaatttggatgaacaagtttcttgtgagtatagagatcacccaaacctaatagagattccaggttgcatctctatttatggcagggatcttccaaatagtgtccaaaataggtctagtttggagtacaagttgttccttcaacgttgccaaagataccgtagtgctcatgatggtatattggtcaatagcttcatggaattggaagaagaagcaacaaaagcaataacccaacatgctaaagggaatgggaattgtagctaccctcctgtgtatccaattgggcctattacacacactgggcctagtgatccaaaaagtgggtgtgaatgtttattgtggttggataaacaaccacctaattcagttctttatgtgtcatttggaagtggtggcacactctgccaagagcagatcaatgaacttgctttagggttggaattgagtaggcacaagtttttgtgggttaatttgagggcaccaaatgatagagcaagtgccacttattttagtgatggcggtttggtggatgatcctttgcattttctaccattagggttcatagagagaaccaaagggcagggtttggttatgtgtggctgggccccacaagttgaggtcctaggtcacaagtcaattggtgcatttttgactcattgtggttggaattcggttttggagagtgtggtgcatggagtgccaatgatggcttggcctttgtttgctgaacaaagaaccaatgcggctttggtaaccgatggattgaaagttgctgtgagaccaaacgttgacactagtggcaatagtgtggtggtgaaggaggaaattgttaagctcataaagagcctcatggaggggttggtgggtgaagaaattcgtaggagaatgaaggaactgcaaaagtttgctgaatgtgctgtgatgaaagatgggtcgtcaacaaggacaatatgcaagttggcacacaagtggaaaagtttgggaagaccttaa

SEQ ID NO: 2 - DNA, 229 bp, Glycine max
gcccttgtttctatcccagctttcagccaccaagtctcaatcctcgagttcgcaaaacgtctccttaatctccacaacaacaccttcaacatcacatgcatcattccaacacttaactcctcttacaatagtgtggtggtgaaggaggaaattgttaagctcataaagagcctcatggaggggttggtgggtgaagaaattcgtaggagaatgaaggaactgcaaaagt

SEQ ID NO: 3 - DNA, 2350 bp, Glycine max
attaggcgcgccatggaatcagcggcaagaacaacaacaacaacaactcatatagcccttgtttctatcccagctttcagccaccaagtctcaatcctcgagttcgcaaaacgtctccttaatctccacaacaacaccttcaacatcacatgcatcattccaacacttaactcctcttacaataacattgccaccaaacccttctttgattccctccctccgaacattcactgcattttcctcccctcagtgtacttcgaggacctaaacaacaatggagtctctgtggagatccaaatccagctctcggtttctcgcgccatgccctccgttagggaaaccctaagatcactctttgatgccaccaacaacgtcgtcgccatcgttgccgatgccatggtcccggaggcgctcgattttggtaaggaactcggcatcttatcctacatctacttcccttgttccacaatgttgctatccctatgccttcattcttcaaatttggatgaacaagtttcttgtgagtatagagatcacccaaacctaatagagattccaggttgcatctctatttatggcagggatcttccaaatagtgtccaaaataggtctagtttggagtacaagttgttccttcaacgttgccaaagataccgtagtgctcatgatggtatattggtcaatagcttcatggaattggaagaagaagcaacaaaagcaataacccaacatgctaaagggaatgggaattgtagctaccctcctgtgtatccaattgggcctattacacacactgggcctagtgatccaaaaagtgggtgtgaatgtttattgtggttggataaacaaccacctaattcagttctttatgtgtcatttggaagtggtggcacactctgccaagagcagatcaatgaacttgctttagggttggaattgagtaggcacaagtttttgtgggttaatttgagggcaccaaatgatagagcaagtgccacttattttagtgatggcggtttggtggatgatcctttgcattttctaccattagggttcatagagagaaccaaagggcagggtttggttatgtgtggctgggccccacaagttgaggtcctaggtcacaagtcaattggtgcatttttgactcattgtggttggaattcggttttggagagtgtggtgcatggagtgccaatgatggcttggcctttgtttgctgaacaaagaaccaatgcggctttggtaaccgatggattgaaagttgctgtgagaccaaacgttgacactagtggcaatagtgtggtggtgaaggaggaaattgttaagctcataaagagcctcatggaggggttggtgggtgaagaaattcgtaggagaatgaaggaactgcaaaagtttgctgaatgtgctgtgatgaaagatgggtcgtcaacaaggacaatatgcaagttggcacacaagtggaaaagtttgggaagaccttaactactagaagattgaatgtggtaatccccttataattaagtttcaactattgtgttttgttttattatttcatattaattaacgtggttgtttgtttggaatgcattaatgtattaaatatttcgttttgcaatttcttaggtaggtatatggtgttttatattgattgaaataacatatgataattaagttcattttatctcattctagtttatatgtctaattatcttcttaatcttaaatttgtatgagataaagtcaactattgtgtattagttttgtcatcatctaatagatttttttatatttaatttttatatactttattcattcataagattctttttaaaaaaattatttattccttttttaagattgtcttttattttttaaatgtattaattatttttttcttacctatctttatttaactattttctctcaaaaatattaataagaaacaattaagtctataaaaagatataaaataatgattttaaaattataatataaataattaacaaatttgatttgattaattaaattaactatttttattaaaaaaacgtaaattaatttaaagaatcttataattagagatgaaagaagtataatgttattttacataaatttgtatttgtttttacatactttaataatttttttgctaagtttaatataataaatctatttaatgaatttaataaataaaactattttttcacataatcctattatataaaaaatataacgtaagtctttaaataataaaataaaagctttctactcatgatgaatatttaaataattttttatctatgttcatcttactctttgcgtcctacttgctgacctaggcgtg

SEQ ID NO: 4 - DNA, 1476 bp, Glycine max
atggaatcagcggcaagaacaacaacaacaacaactcatatagcccttgtttctatcccagctttcagccaccaagtctcaatcctcgagttcgcaaaacgtctccttaatctccacaacaacaccttcaacatcacatgcatcattccaacacttaactcctcttacaataacattgccaccaaacccttctttgattccctccctccgaacattcactgcattttcctcccctcagtgtacttcgaggacctaaacaacaatggagtctctgtggagatccaaatccagctctcggtttctcgcgccatgccctccgttagggaaaccctaagatcactctttgatgccaccaacaacgtcgtcgccatcgttgccgatgccatggtcccggaggcgctcgattttggtaaggaactcggcatcttatcctacatctacttcccttgttccacaatgttgctatccctatgccttcattcttcaaatttggatgaacaagtttcttgtgagtatagagatcacccaaacctaatagagattccaggttgcatctctatttatggcagggatcttccaaatagtgtccaaaataggtctagtttggagtacaagttgttccttcaacgttgccaaagataccgtagtgctcatgatggtatattggtcaatagcttcatggaattggaagaagaagcaacaaaagcaataacccaacatgctaaagggaatgggaattgtagctaccctcctgtgtatccaattgggcctattacacacactgggcctagtgatccaaaaagtgggtgtgaatgtttattgtgattggataaacaaccacctaattcagttctttatgtgtcatttggaagtggtggcacactctgccaagagcagatcaatgaacttgctttagggttggaattgagtaggcacaagtttttgtgggttaatttgagggcaccaaatgatagagcaagtgccacttattttagtgatggcggtttggtggatgatcctttgcattttctaccattagggttcatagagagaaccaaagggcagggtttggttatgtgtggctgggccccacaagttgaggtcctaggtcacaagtcaattggtgcatttttgactcattgtggttggaattcggttttggagagtgtggtgcatggagtgccaatgatggcttggcctttgtttgctgaacaaagaaccaatgcggctttggtaaccgatggattgaaagttgctgtgagaccaaacgttgacactagtggcaatagtgtggtggtgaaggaggaaattgttaagctcataaagagcctcatggaggggttggtgggtgaagaaattcgtaggagaatgaaggaactgcaaaagtttgctgaatgtgctgtgatgaaagatgggtcgtcaacaaggacaatatgcaagttggcacacaagtggaaaagtttgggaagaccttaa

SEQ ID NO: 5 - DNA, 825 bp, Glycine max
atggaatcagcggcaagaacaacaacaacaacaactcatatagcccttgtttctatcccagctttcagccaccaagtctcaatcctcgagttcgcaaaacgtctccttaatctccacaacaacaccttcaacatcacatgcatcattccaacacttaactcctcttacaataacattgccaccaaacccttctttgattccctccctccgaacattcactgcattttcctcccctcagtgtacttcgaggacctaaacaacaatggagtctctgtggagatccaaatccagctctcggtttctcgcgccatgccctccgttagggaaaccctaagatcactctttgatgccaccaacaacgtcgtcgccatcgttgccgatgccatggtcccggaggcgctcgattttggtaaggaactcggcatcttatcctacatctacttcccttgttccacaatgttgctatccctatgccttcattcttcaaatttggatgaacaagtttcttgtgagtatagagatcacccaaacctaatagagattccaggttgcatctctatttatggcagggatcttccaaatagtgtccaaaataggtctagtttggagtacaagttgttccttcaacgttgccaaagataccgtagtgctcatgatggtatattggtcaatagcttcatggaattggaagaagaagcaacaaaagcaataacccaacatgctaaagggaatgggaattgtagctaccctcctgtgtatccaattgggcctattacacacactgggcctagtgatccaaaaagtgggtgtgaatgtttattgtga

SEQ ID NO: 6 - DNA, 2350 bp, Glycine max
attaggcgcgccatggaatcagcggcaagaacaacaacaacaacaactcatatagcccttgtttctatcccagctttcagccaccaagtctcaatcctcgagttcgcaaaacgtctccttaatctccacaacaacaccttcaacatcacatgcatcattccaacacttaactcctcttacaataacattgccaccaaacccttctttgattccctccctccgaacattcactgcattttcctcccctcagtgtacttcgaggacctaaacaacaatggagtctctgtggagatccaaatccagctctcggtttctcgcgccatgccctccgttagggaaaccctaagatcactctttgatgccaccaacaacgtcgtcgccatcgttgccgatgccatggtcccggaggcgctcgattttggtaaggaactcggcatcttatcctacatctacttcccttgttccacaatgttgctatccctatgccttcattcttcaaatttggatgaacaagtttcttgtgagtatagagatcacccaaacctaatagagattccaggttgcatctctatttatggcagggatcttccaaatagtgtccaaaataggtctagtttggagtacaagttgttccttcaacgttgccaaagataccgtagtgctcatgatggtatattggtcaatagcttcatggaattggaagaagaagcaacaaaagcaataacccaacatgctaaagggaatgggaattgtagctaccctcctgtgtatccaattgggcctattacacacactgggcctagtgatccaaaaagtgggtgtgaatgtttattgtgattggataaacaaccacctaattcagttctttatgtgtcatttggaagtggtggcacactctgccaagagcagatcaatgaacttgctttagggttggaattgagtaggcacaagtttttgtgggttaatttgagggcaccaaatgatagagcaagtgccacttattttagtgatggcggtttggtggatgatcctttgcattttctaccattagggttcatagagagaaccaaagggcagggtttggttatgtgtggctgggccccacaagttgaggtcctaggtcacaagtcaattggtgcatttttgactcattgtggttggaattcggttttggagagtgtggtgcatggagtgccaatgatggcttggcctttgtttgctgaacaaagaaccaatgcggctttggtaaccgatggattgaaagttgctgtgagaccaaacgttgacactagtggcaatagtgtggtggtgaaggaggaaattgttaagctcataaagagcctcatggaggggttggtgggtgaagaaattcgtaggagaatgaaggaactgcaaaagtttgctgaatgtgctgtgatgaaagatgggtcgtcaacaaggacaatatgcaagttggcacacaagtggaaaagtttgggaagaccttaactactagaagattgaatgtggtaatccccttataattaagtttcaactattgtgttttgttttattatttcatattaattaacgtggttgtttgtttggaatgcattaatgtattaaatatttcgttttgcaatttcttaggtaggtatatggtgttttatattgattgaaataacatatgataattaagttcattttatctcattctagtttatatgtctaattatcttcttaatcttaaatttgtatgagataaagtcaactattgtgtattagttttgtcatcatctaatagatttttttatatttaatttttatatactttattcattcataagattctttttaaaaaaattatttattccttttttaagattgtcttttattttttaaatgtattaattatttttttcttacctatctttatttaactattttctctcaaaaatattaataagaaacaattaagtctataaaaagatataaaataatgattttaaaattataatataaataattaacaaatttgatttgattaattaaattaactatttttattaaaaaaacgtaaattaatttaaagaatcttataattagagatgaaagaagtataatgttattttacataaatttgtatttgtttttacatactttaataatttttttgctaagtttaatataataaatctatttaatgaatttaataaataaaactattttttcacataatcctattatataaaaaatataacgtaagtctttaaataataaaataaaagctttctactcatgatgaatatttaaataattttttatctatgttcatcttactctttgcgtcctacttgctgacctaggcgtg

SEQ ID NO: 7 - protein, 491 aa, Glycine max
MESAARTTTTTTHIALVSIPAFSHQVSILEFAKRLLNLHNNTFNITCIIPTLNSSYNNIATKPFFDSLPPNIHCIFLPSVYFEDLNNNGVSVEIQIQLSVSRAMPSVRETLRSLFDATNNVVAIVADAMVPEALDFGKELGILSYIYFPCSTMLLSLCLHSSNLDEQVSCEYRDHPNLIEIPGCISIYGRDLPNSVQNRSSLEYKLFLQRCQRYRSAHDGILVNSFMELEEEATKAITQHAKGNGNCSYPPVYPIGPITHTGPSDPKSGCECLLWLDKQPPNSVLYVSFGSGGTLCQEQINELALGLELSRHKFLWVNLRAPNDRASATYFSDGGLVDDPLHFLPLGFIERTKGQGLVMCGWAPQVEVLGHKSIGAFLTHCGWNSVLESVVHGVPMMAWPLFAEQRTNAALVTDGLKVAVRPNVDTSGNSVVVKEEIVKLIKSLMEGLVGEEIRRRMKELQKFAECAVMKDGSSTRTICKLAHKWKSLGRP

SEQ ID NO: 8 - protein, 76 aa, Glycine max
ALVSIPAFSHQVSILEFAKRLLNLHNNTFNITCIIPTLNSSYNSVVVKEEIVKLIKSLMEGLVGEEIRRRMKELQK

SEQ ID NO: 9 - protein, 274 aa, Glycine max
MESAARTTTTTTHIALVSIPAFSHQVSILEFAKRLLNLHNNTFNITCIIPTLNSSYNNIATKPFFDSLPPNIHCIFLPSVYFEDLNNNGVSVEIQIQLSVSRAMPSVRETLRSLFDATNNVVAIVADAMVPEALDFGKELGILSYIYFPCSTMLLSLCLHSSNLDEQVSCEYRDHPNLIEIPGCISIYGRDLPNSVQNRSSLEYKLFLQRCQRYRSAHDGILVNSFMELEEEATKAITQHAKGNGNCSYPPVYPIGPITHTGPSDPKSGCECLL

SEQ ID NO: 10 - DNA, 2500 bp, Glycine max
tcttgattattaattaaaattatttaattacttgataaagaaatattgtaaacttgtgtataaaaattatagaaaaatgggataaattcactttaggctctcatgttagaatttcagtggagaaaatcccttcctaaaaggagtcttgcgattgaaccacaaaacatacccaacttacgtaacagatcaaccaaagatgatttggatatcatttacacaacagatgccctcaatatataaggttgtttttaatagatgggaagagagtacttaaacgtgaaggaagatgaaagtgattatatgaaatattatttttgttcttttatgttttaatattttttttattttattatattaagttctaaaaatttcattttatttttttatattttttaagaattttattttgactaaaacattaattatgttaatagaaaagtgtgatatatttattaacattgattaacacaatgatgtgatatattacatcattttaattgatttcatttgatttttttatgttttttaaatggttttattttagtattttatattttttaaaaggttttattttcagatttgtgaaatgatacattttgatcttaaaaattatatatgatatcaacatctagtaaatgtaattgatggtattctagtaaaactatcacaattttttgtcttagatacctctacaataacttcaataagtatttccctaagcaagtattgtggagggatgtcaaagccatctatctacttgggaaggtaacgtgtcacctgggataaacacgagattgaagattgaatctattgcgacccaatattgatttactgtactaggttttttctatgttgggtgttgtaatatatttaatatttttttattcttttagcatcactaattgacacatatgtataagtatgcacataaataatgtatttataaaataattttcacatgcacaccggcacaagtcaattagtaatgttaaaaaaattgtaaacagaaaatacattacaatattcaactaaaaataattttggaataatgtctccaattcaatttactagactatgttatcatatataatttgttaggatcaacatgtaatattttgcacacttgatggatcaaaatgaatccatttaataaacttaaattatcaataaacttaaattatcaatattataaaatgtaatattcttttaatataagcatcaaaatgaaattatgtaaaatatacgtcattatcacaataaacgctaaagagcacgtcacactttcctgttaattaacacatttaacattttagccacgaaagaggaccaaaacaaaatcttttgaaattataaaaaatcaaaataaaaattttaaaacttcatataccaaaataaataaaaaatactacaatataaaagaccaaaagtaacatttaccctaaaaaaaccaaagccccgtaattttattaaggaaaaatctaaatgtacgcctcatcttttggtgtattgccgtgaaaagtaacgtaaatatcatgaaattttaagttattgtttaatgggtatattgatgtttcctattaggatatgatttttttattattaaaaatagtcattttaaaaccgtaactatttattaggatctaattttaataaagttttcttattttttttacttaatttctaaaattaagcaggttattaaatagtttattaaagagtaaaatcaaagtaaacgtttttatattttattgaaaactaactaaaaacattaatcattttctgttttttaataagcaaataagaagtatactatattgatagcaaaaactgggcagtgaggtcataagaaatttcctcactaacaccacttacatcaggtaggcaaattacaaaacaaaacaccactagtcttaaaaccatatctgctaaagagcccacccttttgccattgcaaaatttagaactagcaacatagctgctaacactaagatctgcagcaccaccaggatctaaatatcaatatttgagatacattctagtatagtgtttttttccccctaaaaagctatacgaactactagaacacggatcaaatggaacagacaaaactttcacatatatagaaattgggtcagtttatttaaatttatttgttaaaatactgcttattttaataaggtaaacaatttttttaatgtgtttatctaaattatttcttgttaaaaaataattttataaaaatatttatattaaagttaattttaaaaaaatttaaacaaactcatttattagtggaccagttagtaattttttgttgttacagtcatacatacattaataataaatgaatatgtacaaccatctaccgctcttgaaagcgaacacatgcagcagtacctttgacgaagttctaagttatacagttgcactttttttatattatattactttgtttcttaccaccattgcaaaaaatcggtctctgcagcaggttttaacc

SEQ ID NO: 11 - DNA, 1859 bp, Artificial Sequence
  synthetic delta endotoxin gene of Bacillus thuringiensis
ggatccaacaatggacaacaatcccaacatcaacgagtgcattccttacaactgcctgagcaaccctgaggttgaggtgctgggtggagaacggattgagactggttacacacctatcgacatctcgttgtcacttacccaattccttttgtcagagttcgtgcccggtgctggattcgtgcttggacttgtcgatatcatttggggaatctttggtccctctcaatgggacgcctttcttgtacagatagagcaacttatcaaccaaaggattgaagagttcgctaggaaccaagccatctcaaggttagaaggcctcagcaacctttaccagatttacgcagaatcttttcgagagtgggaagcagacccgaccaatcctgccttaagagaggagatgcgcattcaattcaatgacatgaacagcgcgctgacgaccgcaattccgctcttcgccgttcagaattaccaagttcctcttttatccgtgtacgtgcaggctgccaacctgcacttgtcggtgctccgcgatgtctccgtgttcggacaacggtggggctttgatgccgcaactatcaatagtcgttataatgatctgactaggcttattggcaactataccgattatgctgttcgctggtacaacacgggtctcgaacgtgtctggggaccggattctagagattgggtcaggtacaaccagttcaggcgagagttgacactaactgtcctagacattgtcgctctctttcccaactacgactctaggcgctacccaatccgtactgtgtcacaattgacccgggaaatctacacaaacccagtcctcgagaacttcgacggtagctttcgaggctcggctcagggcatagagagaagcatcaggtctccacacctgatggacatattgaacagtatcacgatctacaccgatgcgcaccgcggttattactactggtcagggcatcagatcatggcatcacccgttgggttctctggaccagaattcactttcccactttacgggactatgggcaatgcagctccacaacaacgtattgttgctcaactcggtcagggcgtgtatagaaccttgtccagcactctatataggagacctttcaacatcggcatcaacaatcaacaattgtctgtgcttgacgggacagaatttgcctatggaacctcctcaaatctgccatccgctgtctacagaaagagcggaacagttgatagcttggatgagatccctccacagaacaacaacgttccacctaggcaagggtttagccatcgccttagccatgtgtccatgttccgttcaggctttagtaatagcagcgttagtatcatcagagctccgatgttctcttggatacatcgtagtgctgagtttaacaacataattgcatccgatagcattactcagatcccagctgtcaaggggaactttctctttaatggttctgtcatttcaggaccaggattcactggaggcgacttggttaggctgaattcttccggcaacaacatccagaatagagggtatattgaagtgcccattcacttcccatcgacatctaccagatatcgtgttcgtgtaaggtatgcctctgttacccctattcacctcaacgtcaattggggtaattcctccatcttttccaatacagtaccagcgacagctacatccttggataatctccaatctagcgatttcggttacttcgaaagtgccaatgccttcacctcttccctaggtaacatagtaggtgttagaaatttctccggaaccgccggagtgataatcgaccgcttcgaattcattcccgttactgcaacgctcgagtaatagatct

SEQ ID NO: 12 - protein, 613 aa, Artificial Sequence
  synthetic delta endotoxin gene of Bacillus thurigiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGNFLFNGSVISGPGFTGGDLVRLNSSGNNIQNRGYIEVPIHFPSTSTRYRVRVRYASVTPIHLNVNWGNSSIFSNTVPATATSLDNLQSSDFGYFESANAFTSSLGNIVGVRNFSGTAGVIIDRFEFIPVTATLE

(19) United States
(12) Patent Application Publication
(10) Pub. No.: US 2015/0211019 A1
(43) Pub. Date: Jul. 30, 2015

(54) Compositions and Methods for Increasing Pest Resistance in Plants

(71) Applicant: University of Georgia Research Foundation, Inc., Athens, GA (US)

(72) Inventors: Wayne A. Parrott, Athens, GA (US); Maria A. Ortega, Athens, GA (US); Bo K. Ha, Jeonbuk (KR); John N. All, Athens, GA (US); Henry R. Boerma, Athens, GA (US); Adam L. Bray, Athens, GA (US)

(21) Appl. No.: 14/421,334

(22) PCT Filed: Aug. 13, 2013
(86) PCT No.: PCT/US2013/054632

§ 371 Date: Feb. 12, 2015

Related U.S. Application Data
(60) Provisional application No. 61/779,765, filed on Mar. 13, 2013.
(60) Provisional application No. 61/682,563, filed on Aug. 13, 2012.

(51) Int. Cl. C12N 15/82; C12N 9/10

(52) U.S. Cl. CPC C12N 15/8286; C12N 15/8218; C12N 9/1051

(57) ABSTRACT

Compositions and methods of reducing expression of a flavonoid glucosyltransferase plants, and transgenic and hybrid plants with increased pest resistance are disclosed. The plants can express a polynucleotide that alters, reduces, or silences expression of a flavonoid glucosyltransferase. The flavonoid glucosyltransferase can be Glyma07g14530, or a variant, homolog, or ortholog thereof. Compositions and methods for placing a gene of interest under an expression control sequence of Glyma07g14530, or a fragment thereof, and transgenic and hybrid plants containing one or more herbivory-inducible genes are disclosed. The plants can include a polynucleotide having 50, 100, 150, 250, 500, 750, 1,000, 1,250, 1,500, or 2,000 or more nucleotides of an expression control sequence of Glyma07g14530 operable linked to a nucleic acid encoding a gene of interest. The plants can include one or more transgenes or QTLs that increases insect resistance, for example a Bt transgene, or a Pb, QTL-H, or QTL-G.

### STATEMENT REGARDING FEDERALLY SPONSORED RESEARCH OR DEVELOPMENT

This invention was made with Government Support under Agreement 2012-67013-19456 awarded to Wayne A. Parrott by the United States Department of Agriculture. The Government has certain rights in the invention.

### FIELD OF THE INVENTION

The field of the invention is generally related to compositions and methods for increasing insect resistance in plants.

### BACKGROUND OF THE INVENTION

From the beginning of agriculture, pests have been a major problem for crop production. Crop plants have had to compete with animal pests, plant pathogens and weeds (Oerke, The Journal of Agricultural Science, 144:31-43 (2005)). Food losses to pests has a critical impact, as there are seven billion people on earth (Population Reference Bureau, 2011), from which at least 925 million are undernourished (Food and Agriculture Organization of the United Nations, 20). Despite an approximate annual investment of $40 billion in the worldwide application of pesticides, and the use of biological and non-chemical control methods; prior to harvest pests destroy 35% to 42% of potential crop production. Insect pests are responsible for 14% of this loss (Pimentel, New N.Y.: John Wiley & Sons (1997)). In the United States, annual losses to pests are about 37%, of which 13% is due to insects. Unfortunately, in the last 50 years, despite a more than 10-fold increase in the amount and toxicity of synthetic insecticides, the percentage of yield lost to insects has nearly doubled (Pimentel, Integrated Pest Management 2009). Host plant resistance (HPR) is an effective mechanism to control insect pests, and the planting of resistant cultivars has become crucial in reducing production costs, and the impact of insecticide residues in the food chain and environment.

Plant breeding programs have attempted the introgression of host plant resistance in elite cultivars. From an agricultural perspective, host plant resistance is found in individual genotypes that suffer less damage from certain insects than other genotypes of that crop (See soybean response to corn earworm, CEW). However, these efforts have been hampered by the lack of functional understanding of the genetic basis of resistance to most insects, and by the diverse ways in which insects cause damage to plants. Approximately two-thirds of all known plant-feeding insects are leaf-eating beetles or caterpillars that have evolved mouthparts for chewing, snipping or tearing tissue (Schoonhoven, et al., Insect-plant biology (2005)). Alternatively, piercing-sucking insects, such as thrips and true-bugs, use a tube-like mouthpart to syphon the liquid content of wounded cells. Leafminer insects develop in, and feed on, soft tissue between epidermal cell layers. Aphids, whiteflies, and leafhoppers are able to insert a specialized stylet between cells, then establishing a feeding site in the phloem (Howe and Jander, Annu Rev Plant Biol, 59:41-66 (2008)).

Thirty quantitative trait loci (QTLs) or single genes for resistance to insects have been identified in major crop species (Yencho, et al., Annu Rev Entomol, 45:393-422 (2000)); however an understanding of the molecular basis for insect resistance is lagging, as compared to that achieved for genes that confer resistance to plant pathogens. Of these insect resistance genes, one of the best characterized is the Mi gene from tomato, which confers resistance to root-knot nematode, the potato aphid, and whiteflies, all of which are phloem-sucking insects (Nombela, et al., Mol Plant Microbe Interact, 16:645-49 (2003)). This resistance (R) gene belongs to the nucleotide-binding site-leucine-rich repeat protein family of R genes, which includes many genes involved in the classic gene-for-gene interaction between plant and pathogens. Resistance conferred by R genes is usually highly specific, and the Mi gene provides greater resistance to European isolates of potato aphid than to North American isolates (Goggin, et al., Environ Entomol, 30:101-06 (2001)).

In contrast, much less is known about genes that confer resistance to chewing insects, even though these tend to cause the greatest amount of crop loss. It is known that herbivore-derived compounds may come in contact with the plant during any stage of the insect's life, and elicit defense reactions; these compounds are known as herbivore-associated molecular patterns (HAMPs) (Mithofer and Boland, Plant Physiol, 146:825-831 (2008)). Also, the wounding caused by caterpillars' feeding induces plant genes that are regulated by jasmonic acid (De Vos, et al., Molecular Plant-Microbe Interactions, 18:923-937 (2005); Devoto, et al., Plant Mol Biol, 58:497-513 (2005); Halitschke, et al., Plant Physiol, 131:1894-1902 (2003); Major, et al., New Phytol, 172:617-635 (2006); Ralph, et al., Mol Ecol, 15:1275-297 (2006); Ralph, et al., Plant Cell Environ, 29:1545-570 (2006); Reymond, et al., Plant Cell, 12:707-720 (2000)); and some caterpillar species regurgitate compounds that further induce novel defense pathways in plants (Walling, J Plant Growth Regul, 19:195-216 (2000); Musser, et al., (2002); Weech, et al., Exp Bot, 59:2437-448 (2008)). However, it is important to note that what the vast majority of the current knowledge of plant-insect interactions has been acquired from studies conducted on plant genotypes without any agriculturally relevant resistance to chewing insects. Therefore these observations probably represent a plant's general response to herbivory. The responses from the interaction between leaf-chewing insects and resistant plant genotypes have yet to be characterized.

In major crop species, molecular markers have been used to map the genomic location of genes for insect resistance. The resistance to chewing insects found in some crops is inherited as a quantitative trait, involving one or more QTLs that cause antixenosis (non-preference), and/or antibiosis (negative effects on insect growth). However, plant breeders frequently must look to exotic germplasm (e.g., wild relatives and landraces) as a source of genes conferring resistance to insects, meaning that linkage drag is a problem. Linkage drag refers to the unintentional introgression of undesirable alleles at loci that are linked to a locus with a desirable allele. As a result, historically, there is an inverse relationship between insect resistance and agronomic performance, as is the case with insect-resistance in soybean (Boethel, Global Plant Genetic Resources for Insect-Resistant Crops, (1999); Narvel, et al., Crop Science, 41:1931 (2001). As stated by Lambert and Taylor (1999) “The primary breeding problem in developing insect resistance has been, and continues to be, in achieving yields equal that of existing cultivars”.

Accordingly, it is an object of the invention to provide compositions and methods for improving a plant's resistance to pests including insects, preferably while maintaining a high level of agronomic performance.

### SUMMARY OF THE INVENTION

Compositions and methods for increasing pest resistance in a plant are disclosed. It has been discovered that altered expression of the flavonoid glucosyltransferase protein encoded by Glyma07g14530 in soybean increases pest resistance in plants. Accordingly, compositions and methods of altering, reducing, and inhibiting expression of a flavonoid glucosyltransferase protein in a plant, including but not limited to transgenic plants and hybrids to increase insect resistance compared to a plant of the same species or cultivar that has unaltered expression or wildtype expression of Glyma07g14530 are disclosed. In some embodiments the flavonoid glucosyltransferase protein that imparts insect resistance is a truncated protein or an alternative splice variant relative to the wildtype protein present in susceptible plants Inhibition of the expression of Glyma07g14530 can be accomplished using known techniques such as siRNA, TALENs, CRISPRs, or zinc fingers.

In some embodiments, the transgenic plants include a polynucleotide including an expression control sequence operably linked to a nucleic acid sequence encoding an antisense nucleic acid that targets polynucleotide encoding a flavonoid glucosyltransferase and alters, reduces, or inhibits expression of the flavonoid glucosyltransferase gene product. The flavonoid glucosyltransferase can be Glyma07g14530, or a variant, homolog, or ortholog thereof. In some embodiments, the Glyma07g14530 has a nucleic acid sequence SEQ ID NO: 1, 2, 3, or a complement thereof, or a nucleic acid encoding the polypeptide of SEQ ID NO:7 or 8, or complement thereof. The nucleic acid can silence expression or translation of the target polynucleotide by RNAi, dsRNA, miRNA, siRNA, or transacting small-interfering RNAs (tasiRNA).

It has also been discovered that the expression control sequence of Glyma07g14530 is induced by herbivory. Accordingly, compositions and methods for placing a gene of interest under an expression control sequence of Glyma07g14530, or a fragment thereof, and transgenic plants containing one or more herbivory-inducible genes are disclosed.

In some embodiments, the transgenic plant includes a polynucleotide including an expression control sequence of SEQ ID NO:10, or a fragment thereof comprising 50, 100, 150, 250, 500, 750, 1,000, 1,250, 1,500, or 2,000 or more nucleotides of SEQ ID NO:10 operable linked to a nucleic acid sequence encoding a gene of interest. In a preferred embodiment, the gene of interest is an insect resistance gene, such as a gene that encodes a Bacillus thuringiensis protein, a derivative thereof or a synthetic polypeptide modeled thereon. The Bacillus thuringiensis protein can be CryIAc or synthetic polypeptide modeled thereon, such as SEQ ID NO:12. In some embodiments, the insect resistance gene encodes a polypeptide or protein selected from the group consisting of a lectin, an avidin, an enzyme inhibitor, an amylase inhibitor, a hormone, a pheromone, an antibody, and an immunotoxin.

It has also been discovered that plants with QTL-H, alone, or in combination with other insect resistance imparting genes, insect resistance modulating constructs, insect modulating QTLs including but not limited to those disclosed herein, have increased insect resistance to leaf-chewing and some sucking insects. In particular, it has been discovered that QTL-H imparts resistance to sucking insects, such as Megacopta cribraria, and plants that have QTL-H exhibit reduced insect infestation and increased crop yield compared to control plants without QTL-H. Therefore, compositions, and transgenic and hybrid plants including QTL-H, and methods of use thereof for reducing insect infection and increasing crop yield are also disclosed.

The transgenic and hybrid plants disclosed herein typically have increased pest resistance compared to a non-transgenic plant of the same species or cultivar. The pest resistance can be conferred through antixenosis, antibiosis, tolerance, or combinations thereof. In preferred embodiments, the disclosed plants have an increased resistance to one or more insects including, but not limited to, lesser corn stalk borer, fall army worm, cut worm, soybean looper (SBL), velvetbean caterpillar (VBC), beet armyworm (BAW), corn earworm (CEW), tobacco budworm (TBW), a coleopteran such as the Mexican bean beetle (MBB), and sucking insects such as Megacopta cribraria (also referred to as bean plataspid and kudzu bug) or combinations thereof. In some embodiments, the transgenic or hybrid plants disclosed herein have an increased resistance to deer herbivory. In some embodiments, the plants are soybean plants that have increased resistance to one or more chewing insect, one or more sucking insects such as Megacopta cribraria, or combinations thereof.

In some embodiments, the transgenic plant includes an insect resistance QTL such as Pb, QTL-H, QTL-G, and combinations thereof.

In some embodiments the transgenic or hybrid plant is a soybean, such as Jack, Resnik, Williams 82, Corsoy, Crawford, Hutcheson, Kunitz, Champ, Benning, and Woodruff. In some embodiments, the transgenic or hybrid plant is a cereal crop such as wheat, oat, barley, or rice; a forage such as bahiagrass, dallisgrass, kleingrass, guineagrass, reed canarygrass, orchardgrass, ricegrass, foxtail, or vetch; a legume such as lentil, or chickpea; an oilseed such as canola; a vegetable such as onion or carrot; or a specialty crop such as caraway, hemp, or sesame.

### BRIEF DESCRIPTION OF THE DRAWINGS

FIG. 1 is a series of images of near-isogenic soybean lines grown under field conditions. The images show (A) control, insect “susceptible” plants; (B) insect resistant “QTL-M” plants containing the QTL-M allele; (C) insect resistant “Bt” plant expressing the synthetic Cry1Ac transgene from B. thuringiensis; and (D) insect resistant “QTL-M+Bt” plants containing the QTL-M allele and expressing the synthetic Cry1Ac transgene from B. thuringiensis following exposure to corn earworm (CEW) larvae.

FIG. 2 is an illustration showing the graphical genotype of 82-cM segment of Chromosome 7 (QTL-M) for 15 insect-resistant soybean genotypes. Adapted from Narvel et al., Crop Science, 41:1931 (2001).

FIG. 3 is an illustration showing the PI229358 BAC-clone contig spanning the QTL-M locus in the soybean chromosome 7.

FIG. 4 is an illustration showing the region in chromosome 7 that is required for the insect resistance conferred by QTL-M as determined in Benning RSL lines.

FIG. 5 a diagram showing gene models contained within the 178-kb region on Williams 82. Rectangles above gene models Glyma07g14470 and Glyma07g14530 illustrate the locations of polymorphism between susceptible and resistant genotypes. Stars indicate genes supported by expressed sequence tags (ESTs) and/or RNA sequencing data.

FIG. 6 is an expression profile of Glyma07g14530 in soybean. In the illustration in the top half of the figure, transcripts obtained by RACE-PCR (lines) are compared to ESTs (short arrow) and RNA sequence reads (long arrow) describe at soybase.org. In the bottom half of the figure the expression profiles of Glyma07g14530 in various plant tissues harvested from 10-day-old plants of the susceptible cultivar, Benning, and its isoline Benning^{QTL-M} are shown. A representative result for Glyma07g14530 transcripts obtained by RACE-PCR in insect resistant (“R”) and insect susceptible (“S”) plants before (“0”) and 72 hour (“72”) after infestation with soybean looper (SBL) is also shown.

FIG. 7 is an illustration of the QTL-M mode of action. In the susceptible plants, the Glyma07g14530 is functional in the flavonoid pathway. In the resistant plants, the altered Glyma07g14530 has altered function and disrupts the flavonoid pathway. This disruption leads to a modified leaf-flavonoid profile, which causes antibiosis and antixenosis to leaf-chewing insects.

FIG. 8 is a bar graph showing the % defoliation of, from left to right, insect susceptible control plants (a); QTL-M plants (d); Pb plants (b); Pb+QTL-M+QTL-H+QTL-G plants (c); Pb+QTL-M plants (f); QTL-M+QTL-H+QTL-G plants (e); Pb plants (e) in an antixenosis assay.

FIG. 9 is a bar graph showing the mg/petri dish of, from left to right, insect susceptible control plants (a); QTL-M plants (d); Pb plants (b); Pb+QTL-M plants (e); Pb+QTL-M+QTL-H+QTL-G plants (f); QTL-M+QTL-H+QTL-G plants (d); Pb plants (c) in an antibiosis assay.

FIG. 10 is a line graph showing the % defoliation on soybean isolines containing insect resistant QTLs, in a field-cage experiment.

FIG. 11 is a bar graph showing the average adults per plant of kudzu bugs on various soybean plants: Benning (control), QTL-M, QTL-H, QTL-MH, and QTL-MGH.

FIG. 12 is a bar graph showing the average larvae per plant of kudzu bugs on various soybean plants: Benning (control), QTL-M, QTL-H, QTL-MH, and QTL-MGH.

FIG. 13 is a bar graph showing yield (Bushels/Hectare) of various soybean plants: Benning (control), QTL-M, QTL-H, QTL-G, QTL-MGH, QTL-MG, and QTL-MH.

### DETAILED DESCRIPTION OF THE INVENTION

### I. Definitions

Unless otherwise indicated, the disclosure encompasses conventional techniques of plant breeding, microbiology, cell biology and recombinant DNA, which are within the skill of the art. See, e.g., Sambrook, et al., Molecular Cloning: A Laboratory Manual, 3rd edition (2001); Current Protocols In Molecular Biology [(F. M. Ausubel, et al. eds., (1987)]; Plant Breeding: Principles and Prospects (Plant Breeding, Vol 1) M. D. Hayward, N. O. Bosemark, I. Romagosa; Chapman & Hall, (1993); Coligan, Dunn, Ploegh, Speicher and Wingfeld, eds. (1995) Current Protocols in Protein Science (John Wiley & Sons, Inc.); the series Methods in Enzymology (Academic Press, Inc.): PCR 2: A Practical Approach (M. J. MacPherson, B. D. Hames and G. R. Taylor eds. (1995)].

Unless otherwise noted, technical terms are used according to conventional usage. Definitions of common terms in molecular biology may be found in Lewin, Genes VII, published by Oxford University Press, 2000; Kendrew et al. (eds.), The Encyclopedia of Molecular Biology, published by Wiley-Interscience, 1999; and Robert A. Meyers (ed.), Molecular Biology and Biotechnology, a Comprehensive Desk Reference, published by VCH Publishers, Inc., 1995; Sambrook, et al., Molecular Cloning: A Laboratory Manual 3rd. edition (2001), Cold Spring Harbor Laboratory Press.

To facilitate understanding of the disclosure, the following definitions are provided:

As used herein, the term “pest” or “plant pest” refers to a destructive insect or animal, such as deer and rabbits, that attacks crops.

As used herein, the term “pest resistance” or “pest resistant plant” refers to the consequence of heritable plant qualities that result in a plant being relatively less damaged than a plant without the qualities. A pest resistant plant is typically one that yields more than a susceptible plant when confronted with pest invasion. Resistance of plants is relative and is based on comparison with plants lacking the resistance characters, i.e., susceptible plants. Pest resistant plants typically suppress pest abundance or elevate the damage tolerance level of the plants. A pest resistant plant can alter the relationship a pest has with its plant host. For example, the pest resistance can be antibiosis, antixenosis (non-preference), or tolerance.

As used herein, the term “insect resistance” or “insect resistant plant” refers to pest resistance or pest resistant plant wherein the pest is an insect or insects.

As used herein, the term “pest susceptibility” or “pest susceptible plant” refers to a plant that is not pest resistant.

As used herein, the term “insect susceptibility” or “insect susceptible plant” refers to pest susceptibility or a pest susceptible plant wherein the pest is an insect or insects.

As used herein, the term “antixenosis” refers to a property of a plant that makes it unattractive to some feeding or ovipositing insects. Antixenosis typically affects the biology of the insect so pest abundance and subsequent damage is reduced compared to that which would have occurred if the insect was on a susceptible crop variety. Antibiosis resistance can result in increased mortality or reduced longevity and reproduction of the insect. Antixenosis resistance can cause an insect response when the insect attempts to use the resistant plant for food, oviposition, or shelter. Methods of measuring antixenosis are known in the art and described in the Examples below.

As used herein, the term “antibiosis” refers to an association between a plant and an insect that is detrimental to the insect or an antagonistic association between an insect and a metabolic substances produced by a plant. Antibiosis affects the behavior of an insect pest and usually is expressed as non-preference of the insect for a resistant plant compared with a susceptible plant. Antibiosis resistance can cause an insect response when the insect attempts to use the resistant plant for food, oviposition, or shelter. Methods of measuring antibiosis are known in the art and described in the Examples below.

As used herein, the term “tolerance” refers to a property in which a plant is able to withstand or recover from damage caused by insect pest abundance equal to that damaging a plant without resistance characteristics (susceptible). Tolerance is a plant response to an insect pest. Thus, tolerance resistance differs from antibiosis and antixenosis resistance in how it affects the insect-plant relationship. Tolerant plants can be damaged, but this damage does not affect the plant's ability to survive and reproduce.

The term, “herbivory,” as used herein, refers to the process whereby an organism, such as an insect or an animal, feeds on a plant or a plant-like organism.

The term “plant” is used in its broadest sense. It includes, but is not limited to, any species of woody, ornamental or decorative crop or cereal, and fruit or vegetable plant. It also refers to a plurality of plant cells that are largely differentiated into a structure that is present at any stage of a plant's development. Such structures include, but are not limited to, a fruit, shoot, stem, leaf, flower petal, etc.

The term “non-naturally occurring plant” refers to a plant that does not occur in nature without human intervention. Non-naturally occurring plants include transgenic plants and plants produced by non-transgenic means such as plant breeding.

The term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue may be in planta, in organ culture, tissue culture, or cell culture. The term “plant part” as used herein refers to a plant structure, a plant organ, or a plant tissue.

The term “plant material” refers to leaves, stems, roots, flowers or flower parts, fruits, pollen, egg cells, zygotes, seeds, cuttings, cell or tissue cultures, or any other part or product of a plant.

The term “plant organ” refers to a distinct and visibly structured and differentiated part of a plant such as a root, stem, leaf, flower bud, or embryo.

The term “plant cell” refers to a structural and physiological unit of a plant, comprising a protoplast and a cell wall. The plant cell may be in form of an isolated single cell or a cultured cell, or as a part of higher organized unit such as, for example, a plant tissue, a plant organ, or a whole plant.

The term “plant cell culture” refers to cultures of plant units such as, for example, protoplasts, cell culture cells, cells in plant tissues, pollen, pollen tubes, ovules, embryo sacs, zygotes and embryos at various stages of development.

The term “transgenic plant” refers to a plant or tree that contains recombinant genetic material not normally found in plants or trees of this type and which has been introduced into the plant in question (or into progenitors of the plant) by human manipulation. Thus, a plant that is grown from a plant cell into which recombinant DNA is introduced by transformation is a transgenic plant, as are all offspring of that plant that contain the introduced transgene (whether produced sexually or asexually). It is understood that the term transgenic plant encompasses the entire plant or tree and parts of the plant or tree, for instance grains, seeds, flowers, leaves, roots, fruit, pollen, stems etc.

The term “construct” refers to a recombinant genetic molecule having one or more isolated polynucleotide sequences. Genetic constructs used for transgene expression in a host organism include in the 5′-3′ direction, a promoter sequence; a sequence encoding a gene of interest; and a termination sequence. The construct may also include selectable marker gene(s) and other regulatory elements for expression.

The term “gene” refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions as well as regulatory regions and can include 5′ and 3′ ends.

The term “orthologous genes” or “orthologs” refer to genes that have a similar nucleic acid sequence because they were separated by a speciation event.

As used herein, “polypeptide” refers generally to peptides and proteins having more than about ten amino acids. The polypeptides can be “exogenous,” meaning that they are “heterologous,” i.e., foreign to the host cell being utilized, such as human polypeptide produced by a bacterial cell.

The term “isolated” is meant to describe a compound of interest (e.g., nucleic acids) that is in an environment different from that in which the compound naturally occurs, e.g., separated from its natural milieu such as by concentrating a peptide to a concentration at which it is not found in nature. “Isolated” is meant to include compounds that are within samples that are substantially enriched for the compound of interest and/or in which the compound of interest is partially or substantially purified. Isolated nucleic acids are at least 60% free, preferably 75% free, and most preferably 90% free from other associated components. An “isolated” nucleic acid molecule or polynucleotide is a nucleic acid molecule that is identified and separated from at least one contaminant nucleic acid molecule with which it is ordinarily associated in the natural source. The isolated nucleic can be, for example, free of association with all components with which it is naturally associated. An isolated nucleic acid molecule is other than in the form or setting in which it is found in nature.

As used herein, the term “locus” refers to a specific position along a chromosome or DNA sequence. Depending upon context, a locus could be a gene, a marker, a chromosomal band or a specific sequence of one or more nucleotides.

The term “vector” refers to a replicon, such as a plasmid, phage, or cosmid, into which another DNA segment may be inserted so as to bring about the replication of the inserted segment. The vectors can be expression vectors.

The term “expression vector” refers to a vector that includes one or more expression control sequences.

The term “expression control sequence” refers to a DNA sequence that controls and regulates the transcription and/or translation of another DNA sequence. Control sequences that are suitable for prokaryotes, for example, include a promoter, optionally an operator sequence, a ribosome binding site, and the like. Eukaryotic cells are known to utilize promoters, polyadenylation signals, and enhancers.

The term “promoter” refers to a regulatory nucleic acid sequence, typically located upstream (5′) of a gene or protein coding sequence that, in conjunction with various elements, is responsible for regulating the expression of the gene or protein coding sequence. The promoters suitable for use in the constructs of this disclosure are functional in plants and in host organisms used for expressing the disclosed polynucleotides. Many plant promoters are publicly known. These include constitutive promoters, inducible promoters, tissue- and cell-specific promoters and developmentally-regulated promoters. Exemplary promoters and fusion promoters are described, e.g., in U.S. Pat. No. 6,717,034, which is herein incorporated by reference in its entirety.

A nucleic acid sequence or polynucleotide is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading frame. Linking can be accomplished by ligation at convenient restriction sites. If such sites do not exist, synthetic oligonucleotide adaptors or linkers are used in accordance with conventional practice.

“Transformed,” “transgenic,” “transfected” and “recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g., a bacterium or plant, which does not contain the heterologous nucleic acid molecule.

The term “endogenous” with regard to a nucleic acid refers to nucleic acids normally present in the host.

The term “heterologous” refers to elements occurring where they are not normally found. For example, a promoter may be linked to a heterologous nucleic acid sequence, e.g., a sequence that is not normally found operably linked to the promoter. When used herein to describe a promoter element, heterologous means a promoter element that differs from that normally found in the native promoter, either in sequence, species, or number. For example, a heterologous control element in a promoter sequence may be a control/regulatory element of a different promoter added to enhance promoter control, or an additional control element of the same promoter. The term “heterologous” thus can also encompasses “exogenous” and “non-native” elements.

As used herein, “homologous” means derived from the same species. For example, a homologous trait is any characteristic of organisms that is derived from a common ancestor. Homologous sequences can be orthologous or paralogous. Homologous sequences are orthologous if they were separated by a speciation event: when a species diverges into two separate species, the divergent copies of a single gene in the resulting species are said to be orthologous. Orthologs, or orthologous genes, are genes in different species that are similar to each other because they originated from a common ancestor. Homologous sequences are paralogous if they were separated by a gene duplication event: if a gene in an organism is duplicated to occupy two different positions in the same genome, then the two copies are paralogous.

The term “percent (%) sequence identity” is defined as the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

As used herein, “polypeptide” refers generally to peptides and proteins having more than about ten amino acids. The polypeptides can be “exogenous,” meaning that they are “heterologous,” i.e., foreign to the host cell being utilized, such as human polypeptide produced by a bacterial cell.

The term “stringent hybridization conditions” as used herein mean that hybridization will generally occur if there is at least 95% and preferably at least 97% sequence identity between the probe and the target sequence. Examples of stringent hybridization conditions are overnight incubation in a solution comprising 50% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared carrier DNA such as salmon sperm DNA, followed by washing the hybridization support in 0.1×SSC at approximately 65° C. Other hybridization and wash conditions are well known and are exemplified in Sambrook, et al, Molecular Cloning: A Laboratory Manual, Third Edition, Cold Spring Harbor, N.Y. (2000).

As used herein, a “cultivar” refers to a cultivated variety.

As used herein, “germplasm” refers to one or more phenotypic characteristics, or one or more genes encoding said one or more phenotypic characteristics, capable of being transmitted between generations.

As used herein, the term “progenitor” refers to any of the species, varieties, cultivars, or germplasm, from which a plant is derived.

As used herein, the term “derivative species, germplasm or variety” refers to any plant species, germplasm or variety that is produced using a stated species, variety, cultivar, or germplasm, using standard procedures of sexual hybridization, recombinant DNA technology, tissue culture, mutagenesis, or a combination of any one or more said procedures.

As used herein, the terms “introgression”, “introgressed” and “introgressing” refer to both a natural and artificial process whereby genes of one species, variety or cultivar are moved into the genome of another species, variety or cultivar, by crossing those species. The process may optionally be completed by backcrossing to the recurrent parent.

As used herein, “plant part” or “part of a plant” can include, but is not limited to cuttings, cells, protoplasts, cell tissue cultures, callus (calli), cell clumps, embryos, stamens, pollen, anthers, pistils, ovules, flowers, seed, petals, leaves, stems, and roots.

As used herein, “quantitative trait loci (QTL)” refers a region of DNA that is closely linked to a specific phenotypic trait. Typically, QTLs underlie continuous traits (those traits that vary continuously) as opposed to discrete traits.

As used herein, a “hybrid” is typically derived from one or more crosses between different varieties, germplasms, populations, breeds or cultivars within a single species, between different subspecies within a species, or between different species within a genus. Typically, hybrids between subspecies are referred to as “intra-specific hybrids” and hybrids between different species within a genus are referred to as “interspecific hybrids.”

As used herein, unless otherwise defined, to “have one or more QTLs” or to “have one or more desirable QTLs” means to have at least one allele of the superior genotype of a particular QTL.

As used herein “superior genotype” is the genotype of the species associated with the desired trait or desired quality of the trait between two plants.

### II. Compositions for Increasing Insect Resistance

Plant resistance to leaf-chewing or sucking insects reduces the need for insecticide applications, therefore diminishing production costs and pesticide concerns. In soybean, resistance to a broad range of leaf-chewing insects and some sucking insects such as the bean plataspid (kudzu bug) is found in PI229358. Its resistance is conferred by three quantitative trait loci (QTLs), which reduce plant defoliation (antixenosis) and larval-weight gain (antibiosis). Of the three QTLs, QTL-M, on chromosome 7, has a major effect in antibiosis and antixenosis (Rector et al., TAG Theoretical and Applied Genetics, 96:786-790 (1998)). Although the presence of these three favorable QTLs was first identified in PI229358, other plants are also known to carry these QTLs.

It has been discovered that a gene located within QTL-M is responsible for its ability to confer insect resistance. The gene encodes a flavonoid glucosyltransferase that has altered or reduced activity, or is inactive. It is believed that because the Glyma07g14530 protein is encoded within QTL-M is altered, its substrate(s) are available for alternative modifications by other enzymes, and the product of these modifications has antibiotic and antixenotic activity against chewing and some sucking insects (FIG. 7).

Compositions and methods for modifying insect resistance in plants are provided. In some embodiments, the compositions and methods involve reducing or inhibiting the activity of an endogenous flavonoid glucosyltransferase, or control sequences thereof responsible for insect susceptibility in the plant. In some embodiments, the compositions and methods involve introducing into the plant one or more heterologous genes, or control sequences that promote, increase or activate insect resistance. In some embodiments, a heterologous gene that confers insect resistance is under the control of a promoter sequence that is activated or increased in response to herbivory. Transgenic and hybrid plants with increased resistance to one or more species of leaf-chewing insects, one or more species of sucking insects, or a combination thereof compared to a non-transgenic plant of the same species or cultivar are also disclosed.

A. Flavonoid Glucosyltransferases

Glycosylation is a widespread modification reaction that is often the last step in the biosynthesis of natural components (Heller, et al., The Flavonoids: Advances in Research since 1986 (1994)). In this reaction, glucosyltransferases are responsible for transferring nucleotide-diphosphate-activated sugars to low molecular weight substrates. The substrates (aglycones) include plant hormones, all major classes of plant secondary metabolites and xenobiotics (Vogt, et al., Trends Plant Sci, 5:380-86 (2000)). Glycosylation regulates many properties in the aglycones, including their bioactivity, solubility, and transport properties within cells and throughout the plant. The more than 7000 flavonoids identified in different plant species originate from combinatorial modifications to a common aromatic structure, and glycosylation is one of their most predominant modifications (Gachon et al., 2005).

Flavonoids: (i) are responsible for the pigmentation in flowers (Tanaka, et al., Plant J, 54:733-749 (2008), (ii) provide protection against UV radiation (Izaguirre, et al., Ann Bot, 99:103-09 (2007) and insects (Diaz Napal, et al., Bioresour Technol, 100:3669-673 (2009); Thoison, et al., Phytochemistry, 65:2173-76 (2004), and (iii) act as signaling molecules (Kobayashi, et al., Mol Microbiol, 51:335-347 (2004). Insects landing or walking on a leaf encounter flavonoid aglycones that can accumulate on the leaf surface, before encountering flavonoid glycosides that are stored in vacuoles within the leaf (Simmonds, Phytochemistry, 64:21-30 (2003)). It is generally accepted that flavonoids, along with other plant polyphenols, can play a role in the protection of plants from insects and mammal herbivores (Harborne, et al., Phytochemistry, 55:481-504 (2000); however the mechanisms by which these compounds regulate the insect behavior remains unknown.

It has been discovered that a flavonoid glucosyltransferase is responsible for pest susceptibility in plants. Accordingly, altering, reducing or inhibiting the expression of a functional flavonoid glucosyltransferase in a plant can increase the pest resistance of the plant.

1. Glyma07g14530

In some embodiments, the flavonoid glucosyltransferase is a soybean Glyma07g14530. Polynucleotides having a Glyma07g14530 gene from a soybean plant are disclosed. The term “Glyma07g1430^{susceptible}” refers to a gene that encodes a full-length flavonoid 3-O-glycosyltransferase in soybean. The term “Glyma07g14530^{resistant}” refers to a gene that encodes altered flavonoid 3-O-glycosyltransferase, such as a truncated or alternative splice variant protein, in soybean. Plants that express a functional flavonoid glucosyltransferase, such as the Glyma07g14530^{susceptible} disclosed herein, are susceptible to herbivory, while plants that express an altered flavonoid glucosyltransferase, such as the Glyma07g14530^{resistant} disclosed herein, are resistant to herbivory.

It is understood that where coding sequences for a Glyma07g14530 gene is provided, also provided are the non-coding sequences that are known or can be identified to correspond to the coding sequence that is provided. For example, where a Glyma07g14530 gene is provided, also provided for use in the disclosed compositions and methods is the 5′ untranslated region (UTR), which contains the endogenous promoter for the Glyma07g14530 gene. It is understood that the skilled artisan can identify these sequences with routine skill and experimentation based on the sequences that are provided.

a. Nucleic Acids

i. Glyma07g14530 Susceptible

As discussed in more detail below, Glyma07g14530^{susceptible} produces a short (229 bp) and a long (1,476 bp) transcript in leaves of Benning soybean. The genomic sequence is the same length as the long isoform, therefore, the gene does not contain introns.

In some embodiments, a coding sequence for the long isoform of Glyma07g14530^{susceptible} is

ATGGAATCAG CGGCAAGAAC AACAACAACA ACAACTCATA TAGCCCTTGT TTCTATCCCA
GCTTTCAGCC ACCAAGTCTC AATCCTCGAG TTCGCAAAAC GTCTCCTTAA TCTCCACAAC
AACACCTTCA ACATCACATG CATCATTCCA ACACTTAACT CCTCTTACAA TAACATTGCC
ACCAAACCCT TCTTTGATTC CCTCCCTCCG AACATTCACT GCATTTTCCT CCCCTCAGTG
TACTTCGAGG ACCTAAACAA CAATGGAGTC TCTGTGGAGA TCCAAATCCA GCTCTCGGTT
TCTCGCGCCA TGCCCTCCGT TAGGGAAACC CTAAGATCAC TCTTTGATGC CACCAACAAC
GTCGTCGCCA TCGTTGCCGA TGCCATGGTC CCGGAGGCGC TCGATTTTGG TAAGGAACTC
GGCATCTTAT CCTACATCTA CTTCCCTTGT TCCACAATGT TGCTATCCCT ATGCCTTCAT
TCTTCAAATT TGGATGAACA AGTTTCTTGT GAGTATAGAG ATCACCCAAA CCTAATAGAG
ATTCCAGGTT GCATCTCTAT TTATGGCAGG GATCTTCCAA ATAGTGTCCA AAATAGGTCT
AGTTTGGAGT ACAAGTTGTT CCTTCAACGT TGCCAAAGAT ACCGTAGTGC TCATGATGGT
ATATTGGTCA ATAGCTTCAT GGAATTGGAA GAAGAAGCAA CAAAAGCAAT AACCCAACAT
GCTAAAGGGA ATGGGAATTG TAGCTACCCT CCTGTGTATC CAATTGGGCC TATTACACAC
ACTGGGCCTA GTGATCCAAA AAGTGGGTGT GAATGTTTAT TGTGGTTGGA TAAACAACCA
CCTAATTCAG TTCTTTATGT GTCATTTGGA AGTGGTGGCA CACTCTGCCA AGAGCAGATC
AATGAACTTG CTTTAGGGTT GGAATTGAGT AGGCACAAGT TTTTGTGGGT TAATTTGAGG
GCACCAAATG ATAGAGCAAG TGCCACTTAT TTTAGTGATG GCGGTTTGGT GGATGATCCT
TTGCATTTTC TACCATTAGG GTTCATAGAG AGAACCAAAG GGCAGGGTTT GGTTATGTGT
GGCTGGGCCC CACAAGTTGA GGTCCTAGGT CACAAGTCAA TTGGTGCATT TTTGACTCAT
TGTGGTTGGA ATTCGGTTTT GGAGAGTGTG GTGCATGGAG TGCCAATGAT GGCTTGGCCT
TTGTTTGCTG AACAAAGAAC CAATGCGGCT TTGGTAACCG ATGGATTGAA AGTTGCTGTG
AGACCAAACG TTGACACTAG TGGCAATAGT GTGGTGGTGA AGGAGGAAAT TGTTAAGCTC
ATAAAGAGCC TCATGGAGGG GTTGGTGGGT GAAGAAATTC GTAGGAGAAT GAAGGAACTG
CAAAAGTTTG CTGAATGTGC TGTGATGAAA GATGGGTCGT CAACAAGGAC AATATGCAAG
TTGGCACACA AGTGGAAAAG TTTGGGAAGA CCTTAA

(SEQ ID NO:1, Glyma07g14530 susceptible, Williams 82 [Glycine max hydroquinone glucosyltransferase-like (LOC 100775351), mRNA, NCBI Reference Sequence: XM_{—}003530115.1]) or a variant thereof having at least 90%, 95%, or more sequence identity to SEQ ID NO:1.

In other embodiments, a coding sequence for the short form of Glyma07g14530^{susceptible} is

GCCCTTGTTTCTATCCCAGCTTTCAGCCACCAAGTCTCAATCCTCGAGT
TCGCAAAACGTCTCCTTAATCTCCACAACAACACCTTCAACATCACATG
CATCATTCCAACACTTAACTCCTCTTACAATAGTGTGGTGGTGAAGGAG
GAAATTGTTAAGCTCATAAAGAGCCTCATGGAGGGGTTGGTGGGTGAAG
AAATTCGTAGGAGAATGAAGGAACTGCAAAAGT

(SEQ ID NO:2, Glyma07g14530 susceptible, Williams 82) or a variant thereof having at least 90%, 95%, or more sequence identity to SEQ ID NO:2.

In still other embodiments a genomic sequence, including 5′ and 3′UTR, of Glyma07g14530^{susceptible} is

ATTAGGCGCG CCATGGAATC AGCGGCAAGA ACAACAACAA CAACAACTCA TATAGCCCTT
GTTTCTATCC CAGCTTTCAG CCACCAAGTC TCAATCCTCG AGTTCGCAAA ACGTCTCCTT
AATCTCCACA ACAACACCTT CAACATCACA TGCATCATTC CAACACTTAA CTCCTCTTAC
AATAACATTG CCACCAAACC CTTCTTTGAT TCCCTCCCTC CGAACATTCA CTGCATTTTC
CTCCCCTCAG TGTACTTCGA GGACCTAAAC AACAATGGAG TCTCTGTGGA GATCCAAATC
CAGCTCTCGG TTTCTCGCGC CATGCCCTCC GTTAGGGAAA CCCTAAGATC ACTCTTTGAT
GCCACCAACA ACGTCGTCGC CATCGTTGCC GATGCCATGG TCCCGGAGGC GCTCGATTTT
GGTAAGGAAC TCGGCATCTT ATCCTACATC TACTTCCCTT GTTCCACAAT GTTGCTATCC
CTATGCCTTC ATTCTTCAAA TTTGGATGAA CAAGTTTCTT GTGAGTATAG AGATCACCCA
AACCTAATAG AGATTCCAGG TTGCATCTCT ATTTATGGCA GGGATCTTCC AAATAGTGTC
CAAAATAGGT CTAGTTTGGA GTACAAGTTG TTCCTTCAAC GTTGCCAAAG ATACCGTAGT
GCTCATGATG GTATATTGGT CAATAGCTTC ATGGAATTGG AAGAAGAAGC AACAAAAGCA
ATAACCCAAC ATGCTAAAGG GAATGGGAAT TGTAGCTACC CTCCTGTGTA TCCAATTGGG
CCTATTACAC ACACTGGGCC TAGTGATCCA AAAAGTGGGT GTGAATGTTT ATTGTGGTTG
GATAAACAAC CACCTAATTC AGTTCTTTAT GTGTCATTTG GAAGTGGTGG CACACTCTGC
CAAGAGCAGA TCAATGAACT TGCTTTAGGG TTGGAATTGA GTAGGCACAA GTTTTTGTGG
GTTAATTTGA GGGCACCAAA TGATAGAGCA AGTGCCACTT ATTTTAGTGA TGGCGGTTTG
GTGGATGATC CTTTGCATTT TCTACCATTA GGGTTCATAG AGAGAACCAA AGGGCAGGGT
TTGGTTATGT GTGGCTGGGC CCCACAAGTT GAGGTCCTAG GTCACAAGTC AATTGGTGCA
TTTTTGACTC ATTGTGGTTG GAATTCGGTT TTGGAGAGTG TGGTGCATGG AGTGCCAATG
ATGGCTTGGC CTTTGTTTGC TGAACAAAGA ACCAATGCGG CTTTGGTAAC CGATGGATTG
AAAGTTGCTG TGAGACCAAA CGTTGACACT AGTGGCAATA GTGTGGTGGT GAAGGAGGAA
ATTGTTAAGC TCATAAAGAG CCTCATGGAG GGGTTGGTGG GTGAAGAAAT TCGTAGGAGA
ATGAAGGAAC TGCAAAAGTT TGCTGAATGT GCTGTGATGA AAGATGGGTC GTCAACAAGG
ACAATATGCA AGTTGGCACA CAAGTGGAAA AGTTTGGGAA GACCTTAACT ACTAGAAGAT
TGAATGTGGT AATCCCCTTA TAATTAAGTT TCAACTATTG TGTTTTGTTT TATTATTTCA
TATTAATTAA CGTGGTTGTT TGTTTGGAAT GCATTAATGT ATTAAATATT TCGTTTTGCA
ATTTCTTAGG TAGGTATATG GTGTTTTATA TTGATTGAAA TAACATATGA TAATTAAGTT
CATTTTATCT CATTCTAGTT TATATGTCTA ATTATCTTCT TAATCTTAAA TTTGTATGAG
ATAAAGTCAA CTATTGTGTA TTAGTTTTGT CATCATCTAA TAGATTTTTT TATATTTAAT
TTTTATATAC TTTATTCATT CATAAGATTC TTTTTAAAAA AATTATTTAT TCCTTTTTTA
AGATTGTCTT TTATTTTTTA AATGTATTAA TTATTTTTTT CTTACCTATC TTTATTTAAC
TATTTTCTCT CAAAAATATT AATAAGAAAC AATTAAGTCT ATAAAAAGAT ATAAAATAAT
GATTTTAAAA TTATAATATA AATAATTAAC AAATTTGATT TGATTAATTA AATTAACTAT
TTTTATTAAA AAAACGTAAA TTAATTTAAA GAATCTTATA ATTAGAGATG AAAGAAGTAT
AATGTTATTT TACATAAATT TGTATTTGTT TTTACATACT TTAATAATTT TTTTGCTAAG
TTTAATATAA TAAATCTATT TAATGAATTT AATAAATAAA ACTATTTTTT CACATAATCC
TATTATATAA AAAATATAAC GTAAGTCTTT AAATAATAAA ATAAAAGCTT TCTACTCATG
ATGAATATTT AAATAATTTT TTATCTATGT TCATCTTACT CTTTGCGTCC TACTTGCTGA
CCTAGGCGTG

(SEQ ID NO:3, Glyma07g14530 susceptible, Williams 82) or a variant thereof having at least 90%, 95%, or more sequence identity to SEQ ID NO:3.

Therefore, a polynucleotide is disclosed having a nucleic acid sequence SEQ ID NO: 1, 2, or 3 or a fragment or variant thereof. Also disclosed is a fragment or variant of Glyma07g14530^{susceptible} having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 1, 2, or 3. A fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, 75, 100, or more nucleotides shorter than SEQ ID NO: 1, 2, or 3.

Also disclosed is a polynucleotide that hybridizes under stringent conditions to a polynucleotide having the nucleic acid sequence SEQ ID NO: 1, 2, or 3 or a fragment or variant thereof.

ii. Glyma07g14530 Resistant

Benning soybean containing QTL-M, which express Glyma07g14530^{resistant}, do not express the short isoform of Glyma07g14530. Glyma07g14530^{resistant} contains a SNP unique to insect-resistant soybeans, which produces an altered Glyma07g14530 protein that may be a truncated or alternative splice variant of the wildtype protein. The protein encoded by Glyma07g14530^{resistant} may have altered function or be non-functional.

In some embodiments, a coding sequence for the long isoform of Glyma07g14530^{resistant} is

ATGGAATCAG CGGCAAGAAC AACAACAACA ACAACTCATA TAGCCCTTGT TTCTATCCCA
GCTTTCAGCC ACCAAGTCTC AATCCTCGAG TTCGCAAAAC GTCTCCTTAA TCTCCACAAC
AACACCTTCA ACATCACATG CATCATTCCA ACACTTAACT CCTCTTACAA TAACATTGCC
ACCAAACCCT TCTTTGATTC CCTCCCTCCG AACATTCACT GCATTTTCCT CCCCTCAGTG
TACTTCGAGG ACCTAAACAA CAATGGAGTC TCTGTGGAGA TCCAAATCCA GCTCTCGGTT
TCTCGCGCCA TGCCCTCCGT TAGGGAAACC CTAAGATCAC TCTTTGATGC CACCAACAAC
GTCGTCGCCA TCGTTGCCGA TGCCATGGTC CCGGAGGCGC TCGATTTTGG TAAGGAACTC
GGCATCTTAT CCTACATCTA CTTCCCTTGT TCCACAATGT TGCTATCCCT ATGCCTTCAT
TCTTCAAATT TGGATGAACA AGTTTCTTGT GAGTATAGAG ATCACCCAAA CCTAATAGAG
ATTCCAGGTT GCATCTCTAT TTATGGCAGG GATCTTCCAA ATAGTGTCCA AAATAGGTCT
AGTTTGGAGT ACAAGTTGTT CCTTCAACGT TGCCAAAGAT ACCGTAGTGC TCATGATGGT
ATATTGGTCA ATAGCTTCAT GGAATTGGAA GAAGAAGCAA CAAAAGCAAT AACCCAACAT
GCTAAAGGGA ATGGGAATTG TAGCTACCCT CCTGTGTATC CAATTGGGCC TATTACACAC
ACTGGGCCTA GTGATCCAAA AAGTGGGTGT GAATGTTTAT TGTGATTGGA TAAACAACCA
CCTAATTCAG TTCTTTATGT GTCATTTGGA AGTGGTGGCA CACTCTGCCA AGAGCAGATC
AATGAACTTG CTTTAGGGTT GGAATTGAGT AGGCACAAGT TTTTGTGGGT TAATTTGAGG
GCACCAAATG ATAGAGCAAG TGCCACTTAT TTTAGTGATG GCGGTTTGGT GGATGATCCT
TTGCATTTTC TACCATTAGG GTTCATAGAG AGAACCAAAG GGCAGGGTTT GGTTATGTGT
GGCTGGGCCC CACAAGTTGA GGTCCTAGGT CACAAGTCAA TTGGTGCATT TTTGACTCAT
TGTGGTTGGA ATTCGGTTTT GGAGAGTGTG GTGCATGGAG TGCCAATGAT GGCTTGGCCT
TTGTTTGCTG AACAAAGAAC CAATGCGGCT TTGGTAACCG ATGGATTGAA AGTTGCTGTG
AGACCAAACG TTGACACTAG TGGCAATAGT GTGGTGGTGA AGGAGGAAAT TGTTAAGCTC
ATAAAGAGCC TCATGGAGGG GTTGGTGGGT GAAGAAATTC GTAGGAGAAT GAAGGAACTG
CAAAAGTTTG CTGAATGTGC TGTGATGAAA GATGGGTCGT CAACAAGGAC AATATGCAAG
TTGGCACACA AGTGGAAAAG TTTGGGAAGA CCTTAA

(SEQ ID NO:4, Glyma07g14530^{resistant}), which includes a premature stop codon beginning at nucleotide 834 of SEQ ID NO:4 (bolded and underlined).

Accordingly, in other embodiments, a coding sequence for the long isoform of Glyma07g14530^{resistant} is an altered long isoform having the coding sequence

ATGGAATCAG CGGCAAGAAC AACAACAACA ACAACTCATA TAGCCCTTGT TTCTATCCCA
GCTTTCAGCC ACCAAGTCTC AATCCTCGAG TTCGCAAAAC GTCTCCTTAA TCTCCACAAC
AACACCTTCA ACATCACATG CATCATTCCA ACACTTAACT CCTCTTACAA TAACATTGCC
ACCAAACCCT TCTTTGATTC CCTCCCTCCG AACATTCACT GCATTTTCCT CCCCTCAGTG
TACTTCGAGG ACCTAAACAA CAATGGAGTC TCTGTGGAGA TCCAAATCCA GCTCTCGGTT
TCTCGCGCCA TGCCCTCCGT TAGGGAAACC CTAAGATCAC TCTTTGATGC CACCAACAAC
GTCGTCGCCA TCGTTGCCGA TGCCATGGTC CCGGAGGCGC TCGATTTTGG TAAGGAACTC
GGCATCTTAT CCTACATCTA CTTCCCTTGT TCCACAATGT TGCTATCCCT ATGCCTTCAT
TCTTCAAATT TGGATGAACA AGTTTCTTGT GAGTATAGAG ATCACCCAAA CCTAATAGAG
ATTCCAGGTT GCATCTCTAT TTATGGCAGG GATCTTCCAA ATAGTGTCCA AAATAGGTCT
AGTTTGGAGT ACAAGTTGTT CCTTCAACGT TGCCAAAGAT ACCGTAGTGC TCATGATGGT
ATATTGGTCA ATAGCTTCAT GGAATTGGAA GAAGAAGCAA CAAAAGCAAT AACCCAACAT
GCTAAAGGGA ATGGGAATTG TAGCTACCCT CCTGTGTATC CAATTGGGCC TATTACACAC
ACTGGGCCTA GTGATCCAAA AAGTGGGTGT GAATGTTTAT TGTGA

(SEQ ID NO:5, Glyma07g14530^{resistant}) or a variant thereof having at least 90%, 95%, or more sequence identity to SEQ ID NO:5.

In still other embodiments a genomic sequence, including 5′ and 3′ UTR, of Glyma07g14530^{resistant} is

ATTAGGCGCG CCATGGAATC AGCGGCAAGA ACAACAACAA CAACAACTCA TATAGCCCTT
GTTTCTATCC CAGCTTTCAG CCACCAAGTC TCAATCCTCG AGTTCGCAAA ACGTCTCCTT
AATCTCCACA ACAACACCTT CAACATCACA TGCATCATTC CAACACTTAA CTCCTCTTAC
AATAACATTG CCACCAAACC CTTCTTTGAT TCCCTCCCTC CGAACATTCA CTGCATTTTC
CTCCCCTCAG TGTACTTCGA GGACCTAAAC AACAATGGAG TCTCTGTGGA GATCCAAATC
CAGCTCTCGG TTTCTCGCGC CATGCCCTCC GTTAGGGAAA CCCTAAGATC ACTCTTTGAT
GCCACCAACA ACGTCGTCGC CATCGTTGCC GATGCCATGG TCCCGGAGGC GCTCGATTTT
GGTAAGGAAC TCGGCATCTT ATCCTACATC TACTTCCCTT GTTCCACAAT GTTGCTATCC
CTATGCCTTC ATTCTTCAAA TTTGGATGAA CAAGTTTCTT GTGAGTATAG AGATCACCCA
AACCTAATAG AGATTCCAGG TTGCATCTCT ATTTATGGCA GGGATCTTCC AAATAGTGTC
CAAAATAGGT CTAGTTTGGA GTACAAGTTG TTCCTTCAAC GTTGCCAAAG ATACCGTAGT
GCTCATGATG GTATATTGGT CAATAGCTTC ATGGAATTGG AAGAAGAAGC AACAAAAGCA
ATAACCCAAC ATGCTAAAGG GAATGGGAAT TGTAGCTACC CTCCTGTGTA TCCAATTGGG
CCTATTACAC ACACTGGGCC TAGTGATCCA AAAAGTGGGT GTGAATGTTT ATTGTGATTG
GATAAACAAC CACCTAATTC AGTTCTTTAT GTGTCATTTG GAAGTGGTGG CACACTCTGC
CAAGAGCAGA TCAATGAACT TGCTTTAGGG TTGGAATTGA GTAGGCACAA GTTTTTGTGG
GTTAATTTGA GGGCACCAAA TGATAGAGCA AGTGCCACTT ATTTTAGTGA TGGCGGTTTG
GTGGATGATC CTTTGCATTT TCTACCATTA GGGTTCATAG AGAGAACCAA AGGGCAGGGT
TTGGTTATGT GTGGCTGGGC CCCACAAGTT GAGGTCCTAG GTCACAAGTC AATTGGTGCA
TTTTTGACTC ATTGTGGTTG GAATTCGGTT TTGGAGAGTG TGGTGCATGG AGTGCCAATG
ATGGCTTGGC CTTTGTTTGC TGAACAAAGA ACCAATGCGG CTTTGGTAAC CGATGGATTG
AAAGTTGCTG TGAGACCAAA CGTTGACACT AGTGGCAATA GTGTGGTGGT GAAGGAGGAA
ATTGTTAAGC TCATAAAGAG CCTCATGGAG GGGTTGGTGG GTGAAGAAAT TCGTAGGAGA
ATGAAGGAAC TGCAAAAGTT TGCTGAATGT GCTGTGATGA AAGATGGGTC GTCAACAAGG
ACAATATGCA AGTTGGCACA CAAGTGGAAA AGTTTGGGAA GACCTTAACT ACTAGAAGAT
TGAATGTGGT AATCCCCTTA TAATTAAGTT TCAACTATTG TGTTTTGTTT TATTATTTCA
TATTAATTAA CGTGGTTGTT TGTTTGGAAT GCATTAATGT ATTAAATATT TCGTTTTGCA
ATTTCTTAGG TAGGTATATG GTGTTTTATA TTGATTGAAA TAACATATGA TAATTAAGTT
CATTTTATCT CATTCTAGTT TATATGTCTA ATTATCTTCT TAATCTTAAA TTTGTATGAG
ATAAAGTCAA CTATTGTGTA TTAGTTTTGT CATCATCTAA TAGATTTTTT TATATTTAAT
TTTTATATAC TTTATTCATT CATAAGATTC TTTTTAAAAA AATTATTTAT TCCTTTTTTA
AGATTGTCTT TTATTTTTTA AATGTATTAA TTATTTTTTT CTTACCTATC TTTATTTAAC
TATTTTCTCT CAAAAATATT AATAAGAAAC AATTAAGTCT ATAAAAAGAT ATAAAATAAT
GATTTTAAAA TTATAATATA AATAATTAAC AAATTTGATT TGATTAATTA AATTAACTAT
TTTTATTAAA AAAACGTAAA TTAATTTAAA GAATCTTATA ATTAGAGATG AAAGAAGTAT
AATGTTATTT TACATAAATT TGTATTTGTT TTTACATACT TTAATAATTT TTTTGCTAAG
TTTAATATAA TAAATCTATT TAATGAATTT AATAAATAAA ACTATTTTTT CACATAATCC
TATTATATAA AAAATATAAC GTAAGTCTTT AAATAATAAA ATAAAAGCTT TCTACTCATG
ATGAATATTT AAATAATTTT TTATCTATGT TCATCTTACT CTTTGCGTCC TACTTGCTGA
CCTAGGCGTG

(SEQ ID NO:6, Glyma07g14530^{resistant}) or a variant thereof having at least 90%, 95%, or more sequence identity to SEQ ID NO:6.

Therefore, a polynucleotide is disclosed having a nucleic acid sequence SEQ ID NO: 4, 5, or 6 or a fragment or variant thereof. Also disclosed is a fragment or variant of Glyma07g14530^{resistant} having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 4, 5, or 6. A fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, 75, 100, or more nucleotides shorter than SEQ ID NO: 4, 5, or 6.

b. Polypeptides

i. Glyma07g14530^{Susceptible}

An amino acid sequence encoded by a Glyma07g14530^{susceptible} gene is also disclosed. Thus disclosed is a polypeptide encoded by the nucleic acid sequence of SEQ ID NO: 4, 5, 6 or a fragment or variant thereof. Also disclosed is a polypeptide encoded by a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 4, 5, 6 or a fragment or variant thereof. Also disclosed is a polypeptide encoded by a polynucleotide that hybridizes under stringent conditions to a polynucleotide consisting of the nucleic acid sequence SEQ ID NO: 4, 5, or 6 or a fragment or variant thereof.

A polypeptide that is a fragment or variant of a Glyma07g14530^{susceptible} gene product is also disclosed. Thus, a polypeptide encoded by a polynucleotide having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 4, 5, or 6 is disclosed. The fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, 75, or more amino acids shorter than the polypeptide encoded by the nucleic acid sequence SEQ ID NO: 4, 5, or 6. In some embodiments, the gene product of Glyma07g14530^{susceptible} long isoform includes the amino acid sequence encoded by SEQ ID NO:1

MESAARTTTT TTHIALVSIP AFSHQVSILE FAKRLLNLHN NTFNITCIIP TLNSSYNNIA
TKPFFDSLPP NIHCIFLPSV YFEDLNNNGV SVEIQIQLSV SRAMPSVRET LRSLFDATNN
VVAIVADAMV PEALDFGKEL GILSYIYFPC STMLLSLCLH SSNLDEQVSC EYRDHPNLIE
IPGCISIYGR DLPNSVQNRS SLEYKLFLQR CQRYRSAHDG ILVNSFMELE EEATKAITQH
AKGNGNCSYP PVYPIGPITH TGPSDPKSGC ECLLWLDKQP PNSVLYVSFG SGGTLCQEQI
NELALGLELS RHKFLWVNLR APNDRASATY FSDGGLVDDP LHFLPLGFIE RTKGQGLVMC
GWAPQVEVLG HKSIGAFLTH CGWNSVLESV VHGVPMMAWP LFAEQRTNAA LVTDGLKVAV
RPNVDTSGNS VVVKEEIVKL IKSLMEGLVG EEIRRRMKEL QKFAECAVMK DGSSTRTICK
LAHKWKSLGR P

(SEQ ID NO:7) or a variant thereof having one or more conservative amino acid substitutions and at least 90%, 95%, or more sequence identity compared to SEQ ID NO:7.

In other embodiments, the gene product of Glyma07g14530^{susceptible} short isoform includes the amino acid sequence encoded by SEQ ID NO:2 ALVSIPAFSHQVSILEFAKRLLNLHNNTFNITCIIPTLNSSYNSVVVKEEIVKLIKSLMEGLVGEEIR RRMKELQK (SEQ ID NO:8) or a variant thereof having one or more conservative amino acid substitutions and at least 90%, 95%, or more sequence identity compared to SEQ ID NO:8.

A polypeptide is therefore disclosed having the amino acid sequence SEQ ID NO: 7, 8, or a fragment or variant thereof. A polypeptide having an amino acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 7 or 8 is also disclosed.

A polypeptide that is a fragment or variant of the Glyma07g14530^{susceptible} protein including the amino acid sequence SEQ ID NO: 7 or 8 is also disclosed. A polypeptide having an amino acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to a fragment of 7 or 8 is disclosed. The fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, or 75 amino acids shorter than SEQ ID NO: 7 or 8.

Also disclosed are polynucleotides encoding the amino acid sequence SEQ ID NO: 7, 8, or fragments or variants thereof.

ii. Glyma07g14530^{resistant}

An amino acid sequence encoding a Glyma07g14530^{resistant} gene product is also disclosed. The polypeptide is encoded by the nucleic acid sequence of SEQ ID NO: 4, 5, 6 or a fragment or variant thereof. Also disclosed is a polypeptide encoded by a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 4, 5, 6 or a fragment or variant thereof. Also disclosed is a polypeptide encoded by a polynucleotide that hybridizes under stringent conditions to a polynucleotide consisting of the nucleic acid sequence SEQ ID NO: 4, 5, 6 or a fragment or variant thereof.

A polypeptide that is a fragment or variant of a Glyma07g14530^{resistant} gene product is also disclosed. Thus, a polypeptide encoded by a polynucleotide having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 4, 5, or 6 is disclosed. The fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, 75, or more amino acids shorter than the polypeptide encoded by the nucleic acid sequence SEQ ID NO: 4, 5, or 6.

In some embodiments, the gene product of Glyma07g14530^{resist}′″ altered long isoform includes the amino acid sequence encoded by SEQ ID NO:5

MESAARTTTT TTHIALVSIP AFSHQVSILE FAKRLLNLHN NTFNITCIIP TLNSSYNNIA
TKPFFDSLPP NIHCIFLPSV YFEDLNNNGV SVEIQIQLSV SRAMPSVRET LRSLFDATNN
VVAIVADAMV PEALDFGKEL GILSYIYFPC STMLLSLCLH SSNLDEQVSC EYRDHPNLIE
IPGCISIYGR DLPNSVQNRS SLEYKLFLQR CQRYRSAHDG ILVNSFMELE EEATKAITQH
AKGNGNCSYP PVYPIGPITH TGPSDPKSGC ECLL

(SEQ ID NO:9) or a variant thereof having one or more conservative amino acid substitutions and at least 90%, 95%, or more sequence identity compared to SEQ ID NO:9.

A polypeptide is therefore disclosed having the amino acid sequence SEQ ID NO: 9 or a fragment or variant thereof. A polypeptide having an amino acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 9 is also disclosed.

A polypeptide that is a fragment or variant of Glyma07g14530^{resistant} protein including the amino acid sequence SEQ ID NO: 9 is also disclosed. A polypeptide having an amino acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to a fragment of 9 is disclosed. The fragment can be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 50, or 75 amino acids shorter than SEQ ID NO: 9.

Also disclosed are polynucleotides encoding the amino acid sequence SEQ ID NO: 9 or fragments or variants thereof.

2. Homologs and Orthologs of Glyma07g14530

The Glyma07g14530 protein is homologous or orthologous to reported flavonoid glucosyltransferases from several plant species, including A. thaliana, Medicago truncatula, and kudzu (He et al., Planta, 233, 843-855, (2011)). Accordingly, in some embodiments, the flavonoid glucosyltransferase is a homolog or ortholog of Glyma07g14530^{susceptible} For example the flavonoid glucosyltransferase can be a hydroquinone glucosyltransferase from Arabidopsis thaliana, see for example, Arabidopsis thaliana hydroquinone glucosyltransferase (GT72B1) mRNA, complete cds NCBI Reference Sequence: NM_{—}116337.2.

In other embodiments, the flavonoid glucosyltransferase is a hydroquinone glucosyltransferase from Medicago truncatula, see for example, Medicago truncatula Hydroquinone glucosyltransferase (MTR_{—}7g047230) mRNA, complete cds NCBI Reference Sequence: XM_{—}003622594.1.

In still other embodiments, the flavonoid glucosyltransferase is a hydroquinone glucosyltransferase from Pueraria lobata, see for example, Pueraria montana var. lobata glycosyltransferase GT07002 mRNA, complete cds GenBank: HQ219043.1.

B. Herbivory Inducible Control Sequences

It has also been discovered that expression of Glyma07g14530 in soybean is induced by herbivory. Accordingly, the control sequences that control expression of Glyma07g14530 can be used to promote herbivory-induced expression of a gene of interest. In some embodiments, the control sequences include genomic untranslated regions 5′ of Glyma07g14530, 3′ of Glyma07g14530, or combinations thereof.

In other embodiments, the control sequence that controls expression of Glyma07g14530 include 50, 100, 150, 250, 500, 750, 1,000, 1,250, 1,500, 2,500, 5,000 nucleotides 5′ of Glyma07g14530, In some embodiments, the control sequence ends immediately before the ATG start codon of Glyma07g14530. For example, the control sequence that controls expression of Glyma07g14530 can include 50, 100, 150, 250, 500, 750, 1,000, 1,250, 1,500, 2,500, 5,000 nucleotides of the nucleic acid sequence

TCTTGATTAT TAATTAAAAT TATTTAATTA CTTGATAAAG AAATATTGTA AACTTGTGTA
TAAAAATTAT AGAAAAATGG GATAAATTCA CTTTAGGCTC TCATGTTAGA ATTTCAGTGG
AGAAAATCCC TTCCTAAAAG GAGTCTTGCG ATTGAACCAC AAAACATACC CAACTTACGT
AACAGATCAA CCAAAGATGA TTTGGATATC ATTTACACAA CAGATGCCCT CAATATATAA
GGTTGTTTTT AATAGATGGG AAGAGAGTAC TTAAACGTGA AGGAAGATGA AAGTGATTAT
ATGAAATATT ATTTTTGTTC TTTTATGTTT TAATATTTTT TTTATTTTAT TATATTAAGT
TCTAAAAATT TCATTTTATT TTTTTATATT TTTTAAGAAT TTTATTTTGA CTAAAACATT
AATTATGTTA ATAGAAAAGT GTGATATATT TATTAACATT GATTAACACA ATGATGTGAT
ATATTACATC ATTTTAATTG ATTTCATTTG ATTTTTTTAT GTTTTTTAAA TGGTTTTATT
TTAGTATTTT ATATTTTTTA AAAGGTTTTA TTTTCAGATT TGTGAAATGA TACATTTTGA
TCTTAAAAAT TATATATGAT ATCAACATCT AGTAAATGTA ATTGATGGTA TTCTAGTAAA
ACTATCACAA TTTTTTGTCT TAGATACCTC TACAATAACT TCAATAAGTA TTTCCCTAAG
CAAGTATTGT GGAGGGATGT CAAAGCCATC TATCTACTTG GGAAGGTAAC GTGTCACCTG
GGATAAACAC GAGATTGAAG ATTGAATCTA TTGCGACCCA ATATTGATTT ACTGTACTAG
GTTTTTTCTA TGTTGGGTGT TGTAATATAT TTAATATTTT TTTATTCTTT TAGCATCACT
AATTGACACA TATGTATAAG TATGCACATA AATAATGTAT TTATAAAATA ATTTTCACAT
GCACACCGGC ACAAGTCAAT TAGTAATGTT AAAAAAATTG TAAACAGAAA ATACATTACA
ATATTCAACT AAAAATAATT TTGGAATAAT GTCTCCAATT CAATTTACTA GACTATGTTA
TCATATATAA TTTGTTAGGA TCAACATGTA ATATTTTGCA CACTTGATGG ATCAAAATGA
ATCCATTTAA TAAACTTAAA TTATCAATAA ACTTAAATTA TCAATATTAT AAAATGTAAT
ATTCTTTTAA TATAAGCATC AAAATGAAAT TATGTAAAAT ATACGTCATT ATCACAATAA
ACGCTAAAGA GCACGTCACA CTTTCCTGTT AATTAACACA TTTAACATTT TAGCCACGAA
AGAGGACCAA AACAAAATCT TTTGAAATTA TAAAAAATCA AAATAAAAAT TTTAAAACTT
CATATACCAA AATAAATAAA AAATACTACA ATATAAAAGA CCAAAAGTAA CATTTACCCT
AAAAAAACCA AAGCCCCGTA ATTTTATTAA GGAAAAATCT AAATGTACGC CTCATCTTTT
GGTGTATTGC CGTGAAAAGT AACGTAAATA TCATGAAATT TTAAGTTATT GTTTAATGGG
TATATTGATG TTTCCTATTA GGATATGATT TTTTTATTAT TAAAAATAGT CATTTTAAAA
CCGTAACTAT TTATTAGGAT CTAATTTTAA TAAAGTTTTC TTATTTTTTT TACTTAATTT
CTAAAATTAA GCAGGTTATT AAATAGTTTA TTAAAGAGTA AAATCAAAGT AAACGTTTTT
ATATTTTATT GAAAACTAAC TAAAAACATT AATCATTTTC TGTTTTTTAA TAAGCAAATA
AGAAGTATAC TATATTGATA GCAAAAACTG GGCAGTGAGG TCATAAGAAA TTTCCTCACT
AACACCACTT ACATCAGGTA GGCAAATTAC AAAACAAAAC ACCACTAGTC TTAAAACCAT
ATCTGCTAAA GAGCCCACCC TTTTGCCATT GCAAAATTTA GAACTAGCAA CATAGCTGCT
AACACTAAGA TCTGCAGCAC CACCAGGATC TAAATATCAA TATTTGAGAT ACATTCTAGT
ATAGTGTTTT TTTCCCCCTA AAAAGCTATA CGAACTACTA GAACACGGAT CAAATGGAAC
AGACAAAACT TTCACATATA TAGAAATTGG GTCAGTTTAT TTAAATTTAT TTGTTAAAAT
ACTGCTTATT TTAATAAGGT AAACAATTTT TTTAATGTGT TTATCTAAAT TATTTCTTGT
TAAAAAATAA TTTTATAAAA ATATTTATAT TAAAGTTAAT TTTAAAAAAA TTTAAACAAA
CTCATTTATT AGTGGACCAG TTAGTAATTT TTTGTTGTTA CAGTCATACA TACATTAATA
ATAAATGAAT ATGTACAACC ATCTACCGCT CTTGAAAGCG AACACATGCA GCAGTACCTT
TGACGAAGTT CTAAGTTATA CAGTTGCACT TTTTTTATAT TATATTACTT TGTTTCTTAC
CACCATTGCA AAAAATCGGT CTCTGCAGCA GGTTTTAACC

(SEQ ID NO:10) or a variant thereof having at least 90%, 95%, or more sequence identity to SEQ ID NO:10.

Therefore, a polynucleotide is disclosed having a nucleic acid sequence SEQ ID NO:10 or a fragment or variant thereof. Also disclosed is a fragment or variant of control sequence having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO: 10. A fragment can be at least 50, 100, 150, 250, 500, 750, 1,000, 1,250, 1,500, 2,000, 2,100, 2,200, 2,250, 2,400, 2,450 or more nucleotides shorter than SEQ ID NO: 10.

### III. Methods for Increasing Pest Resistance

Methods of modulating pest resistance in plants are also disclosed. The methods can be used, for example, to increase a plant's resistance to pests such as insects or parasites. The methods can include, for example, altering, reducing or inhibiting the expression of a functional flavonoid glucosyltransferase in a plant, expressing a pest resistance gene or QTL in a plant, or combinations thereof. In some embodiments, the pest resistance gene is placed under the control of an herbivory inducible expression control sequence, or sequences.

A. Methods of Modulating Functional Flavonoid Glucosyltransferase Expression

Methods of modulating pest resistance can include modulating the expression of a flavonoid glucosyltransferase, such as Glyma07g14530, or a fragment, variant, ortholog, or homolog thereof.

In some embodiments, pest resistance is increased in a plant by altering or decreasing the expression of a functional flavonoid glucosyltransferase gene or gene product, for example, a functional Glyma07g14530 protein or a functional fragment, variant, ortholog or homolog thereof.

In other embodiments, the method involves inhibiting flavonoid glucosyltransferase activity in a plant. In still other embodiments, the method involves engineering a transgenic plant to alter, reduce or inhibit expression of a flavonoid glucosyltransferase gene or gene product, or translation of a flavonoid glucosyltransferase protein. For example, the method can involve introducing to the plant a composition that alters or silences gene expression. The composition can include an antisense nucleic acid that encodes RNAi, dsRNA, miRNA, or siRNA that targets the flavonoid glucosyltransferase in the plant and prevents or inhibits translation of the encoded protein, or alters expression of the protein, for example by producing an alternative splice variant. In some embodiments, the compositions mediate production by the plant of transacting small-interfering RNAs (tasiRNA) against the flavonoid glucosyltransferase. In still other embodiments, the method involves introducing into the plant a composition that binds to the protein encoded by the flavonoid glucosyltransferase and inhibits one or more of the protein's activities.

Expression of the flavonoid glucosyltransferase can be inhibited using known techniques such as Zing fingers, TALENs, and CRSPRs.

In still other embodiments, the method involves introducing into the plant or plant cell a nucleic acid sequence that alters or silences expression of a flavonoid glucosyltransferase in the plant. In a preferred embodiment, the flavonoid glucosyltransferase is Glyma07g14530, or an ortholog or homolog thereof. Preferably, the nucleic acid is operably linked to an expression control sequence. The expression control sequence can be an herbivory inducible control sequence, for example the endogenous control sequence of Glyma07g14530. The expression control sequence can be a heterologous control sequence. Selection of this control sequence can be used to select the amount of gene-silencing nucleic acid expressed and therefore control expression of the flavonoid glucosyltransferase in the plant. As a result of this method, the transgenic plant preferably has lower flavonoid glucosyltransferase activity compared to a control (e.g., wild-type) plant of the same species or cultivar. In some embodiments, the nucleic acid can silence a polynucleotide having the nucleic acid sequence SEQ ID NO: 1, 2, or 3, or a nucleic acid encoding the polypeptide of SEQ ID NO: 7 or 8 or fragments, variants, orthologs, or homologs thereof.

In one embodiment, the method of reducing expression of a flavonoid glucosyltransferase in a plant includes transfecting the plant with compositions that induce production of tasiRNA that mediate alteration or silencing of the flavonoid glucosyltransferase expression. The method can include introducing a polynucleotide including an miRNA target sequence operably linked to a sequence encoding the flavonoid glucosyltransferase into a cell. The miRNA target sequence operably linked to a sequence encoding the flavonoid glucosyltransferase can be transcribed in vitro and transiently transfected into the cell. Such methods are known in the art, see for example U.S. Published Application No. 2011/0165133 which is incorporated by reference in its entirety.

The miRNA target sequence can be operably linked to the flavonoid glucosyltransferase are expressed from an expression construct. In preferred embodiments, the miRNA target sequence operably linked to a sequence encoding the flavonoid glucosyltransferase is inserted into a plant vector, which can be transformed into the plant cell. The miRNA target sequence can also be operably linked to a sequence encoding a polynucleotide of interest is integrated into the nuclear or an organelle genome of the plant. In the some embodiments the construct is expressed extra-chromosomally.

To induce gene silencing in a plant, the miRNA target sequence operably linked to the flavonoid glucosyltransferase is typically co-expressed with an miRNA specific for the target miRNA sequence. Similar to the construct containing the miRNA target sequence operably linked to the flavonoid glucosyltransferase, the miRNA can be transiently transfected into the cell, or expressed from a vector. The miRNA can be integrated into the nuclear genome or an organelle genome of the plant, or expressed extra-chromosomally. In some embodiments, the miRNA is an endogenous miRNA that is expressed, or can be induced to be expressed by the plant cell. The miRNA can also be a heterologous miRNA.

As discussed above, when co-expressed, the miRNA binds to the miRNA target sequence and induce generation of tasiRNA which can mediate gene silencing of the flavonoid glucosyltransferase. tasiRNA can be used to create transgenic plants with inducible or stable silencing the flavonoid glucosyltransferase expression.

Methods of interfering with the non-coding segments of a flavonoid glucosyltransferase such as Glyma07g14530 can be used to modulate the pest resistance. Deleting or altering some or all of the non-coding segments or inserting additional nucleotides into the non-coding segments can be effective to increase resistance to pests. Deleting, mutating, or inserting nucleotides in one or more of the flavonoid glucosyltransferase expression control sequences, for example, the Glyma07g14530 control sequences disclosed herein can decrease the expression of the flavonoid glucosyltransferase. Therefore, in some embodiments deleting or mutating nucleotides in flavonoid glucosyltransferase expression control sequence can shift the plant from pest susceptible to pest resistant. For example, in some embodiments insertions, mutations, or deletions are introduced into a polynucleotide having SEQ ID NO: 10 or a functional fragment, variant, or complement thereof to reduce the herbivory sensitivity of the expression control sequence.

Inhibiting the regulatory function of the non-coding segments can also be used to modulate a flavonoid glucosyltransferase. For instance, inhibiting or preventing the interaction of one or more of the non-coding segments with another nucleic acid sequence or protein. The additional nucleotides can be dependent on or independent of a functional copy of the flavonoid glucosyltransferase gene.

Methods of modifying the pest resistance of a plant can include replacing or supplementing the endogenous control sequences of a flavonoid glucosyltransferase with heterologous control sequences. The expression control sequences of the flavonoid glucosyltransferase can be altered or replaced with an expression control sequence that reduces induction during herbivory, but wherein expression of the flavonoid glucosyltransferase can be activated or induced during other periods, for example in the absence of insect infestation.

B. Methods of Modulating Insect Resistance Using Herbivory Inducible Control Sequences

The methods of modulating pest resistance in plants disclosed herein can include inducing or increasing expression of pest resistance gene in a plant. In some embodiments, the pest resistance gene is placed under the control of an herbivory inducible expression control sequence, or sequences.

Herbivory inducible expression control sequences include, but are not limited to the control sequences of Glyma07g14530, or a fragment thereof. For example, the expression control sequence can include 50, 100, 150, 250, 500, 750, 1,000, 1,250, 1,500, 2,000, or 2,500 nucleotides of the nucleic acid sequence SEQ ID NO:10.

The pest resistance gene, for example an insect resistance gene, can be an endogenous or heterologous gene. The pest resistance gene can impart resistance through antixenosis, antibiosis, tolerance, or combinations thereof.

One example of an insect resistance gene is a Bacillus thuringiensis protein, a derivative thereof or a synthetic polypeptide modeled thereon. See, for example, Geiser et al. (Gene, 48(1):109-118, 1986), which describes the cloning and nucleotide sequence of a Bacillus thuringiensis δ-endotoxin gene (also referred to herein as cryIAc gene), and Stewart, Jr., et al., Plant Physio., 112:121-129 (1996), which describes an insect resistance transgenic soybean plant expressing the δ-endotoxin gene. Moreover, DNA molecules encoding δ-endotoxin genes can be purchased from the American Type Culture Collection, Manassas, Va., for example, under ATCC Accession Nos. 40098, 67136, 31995 and 31998.

In some embodiments, the insect resistant gene is a synthetic Bt crylAc which is truncated, or codon optimized for expression in plants, or a combination thereof. The synthetic Bt crylAc can be a polynucleotide having the nucleic acid sequence

| | |
|---|---|
| 1 | GGATCCAACA ATGGACAACA ATCCCAACAT CAACGAGTGC ATTCCTTACA ACTGCCTGAG |
| 61 | CAACCCTGAG GTTGAGGTGC TGGGTGGAGA ACGGATTGAG ACTGGTTACA CACCTATCGA |
| 121 | CATCTCGTTG TCACTTACCC AATTCCTTTT GTCAGAGTTC GTGCCCGGTG CTGGATTCGT |
| 181 | GCTTGGACTT GTCGATATCA TTTGGGGAAT CTTTGGTCCC TCTCAATGGG ACGCCTTTCT |
| 241 | TGTACAGATA GAGCAACTTA TCAACCAAAG GATTGAAGAG TTCGCTAGGA ACCAAGCCAT |
| 301 | CTCAAGGTTA GAAGGCCTCA GCAACCTTTA CCAGATTTAC GCAGAATCTT TTCGAGAGTG |
| 361 | GGAAGCAGAC CCGACCAATC CTGCCTTAAG AGAGGAGATG CGCATTCAAT TCAATGACAT |
| 421 | GAACAGCGCG CTGACGACCG CAATTCCGCT CTTCGCCGTT CAGAATTACC AAGTTCCTCT |
| 481 | TTTATCCGTG TACGTGCAGG CTGCCAACCT GCACTTGTCG GTGCTCCGCG ATGTCTCCGT |
| 541 | GTTCGGACAA CGGTGGGGCT TTGATGCCGC AACTATCAAT AGTCGTTATA ATGATCTGAC |
| 601 | TAGGCTTATT GGCAACTATA CCGATTATGC TGTTCGCTGG TACAACACGG GTCTCGAACG |
| 661 | TGTCTGGGGA CCGGATTCTA GAGATTGGGT CAGGTACAAC CAGTTCAGGC GAGAGTTGAC |
| 721 | ACTAACTGTC CTAGACATTG TCGCTCTCTT TCCCAACTAC GACTCTAGGC GCTACCCAAT |
| 781 | CCGTACTGTG TCACAATTGA CCCGGGAAAT CTACACAAAC CCAGTCCTCG AGAACTTCGA |
| 841 | CGGTAGCTTT CGAGGCTCGG CTCAGGGCAT AGAGAGAAGC ATCAGGTCTC CACACCTGAT |
| 901 | GGACATATTG AACAGTATCA CGATCTACAC CGATGCGCAC CGCGGTTATT ACTACTGGTC |
| 961 | AGGGCATCAG ATCATGGCAT CACCCGTTGG GTTCTCTGGA CCAGAATTCA CTTTCCCACT |
| 1021 | TTACGGGACT ATGGGCAATG CAGCTCCACA ACAACGTATT GTTGCTCAAC TCGGTCAGGG |
| 1081 | CGTGTATAGA ACCTTGTCCA GCACTCTATA TAGGAGACCT TTCAACATCG GCATCAACAA |
| 1141 | TCAACAATTG TCTGTGCTTG ACGGGACAGA ATTTGCCTAT GGAACCTCCT CAAATCTGCC |
| 1201 | ATCCGCTGTC TACAGAAAGA GCGGAACAGT TGATAGCTTG GATGAGATCC CTCCACAGAA |
| 1261 | CAACAACGTT CCACCTAGGC AAGGGTTTAG CCATCGCCTT AGCCATGTGT CCATGTTCCG |
| 1321 | TTCAGGCTTT AGTAATAGCA GCGTTAGTAT CATCAGAGCT CCGATGTTCT CTTGGATACA |
| 1381 | TCGTAGTGCT GAGTTTAACA ACATAATTGC ATCCGATAGC ATTACTCAGA TCCCAGCTGT |
| 1441 | CAAGGGGAAC TTTCTCTTTA ATGGTTCTGT CATTTCAGGA CCAGGATTCA CTGGAGGCGA |
| 1501 | CTTGGTTAGG CTGAATTCTT CCGGCAACAA CATCCAGAAT AGAGGGTATA TTGAAGTGCC |
| 1561 | CATTCACTTC CCATCGACAT CTACCAGATA TCGTGTTCGT GTAAGGTATG CCTCTGTTAC |
| 1621 | CCCTATTCAC CTCAACGTCA ATTGGGGTAA TTCCTCCATC TTTTCCAATA CAGTACCAGC |
| 1681 | GACAGCTACA TCCTTGGATA ATCTCCAATC TAGCGATTTC GGTTACTTCG AAAGTGCCAA |
| 1741 | TGCCTTCACC TCTTCCCTAG GTAACATAGT AGGTGTTAGA AATTTCTCCG GAACCGCCGG |
| 1801 | AGTGATAATC GACCGCTTCG AATTCATTCC CGTTACTGCA ACGCTCGAGT AATAGATCT |

(SEQ ID NO:11) or a variant thereof having at least 90%, 95%, or more sequence identity to SEQ ID NO:11.

In other embodiments the synthetic Bt crylAc, is a polynucleotide encoding a polypeptide having the amino acid sequence

MDNNPNINEC IPYNCLSNPE VEVLGGERIE TGYTPIDISL SLTQFLLSEF VPGAGFVLGL
VDIIWGIFGP SQWDAFLVQI EQLINQRIEE FARNQAISRL EGLSNLYQIY AESFREWEAD
PTNPALREEM RIQFNDMNSA LTTAIPLFAV QNYQVPLLSV YVQAANLHLS VLRDVSVFGQ
RWGFDAATIN SRYNDLTRLI GNYTDYAVRW YNTGLERVWG PDSRDWVRYN QFRRELTLTV
LDIVALFPNY DSRRYPIRTV SQLTREIYTN PVLENFDGSF RGSAQGIERS IRSPHLMDIL
NSITIYTDAH RGYYYWSGHQ IMASPVGFSG PEFTFPLYGT MGNAAPQQRI VAQLGQGVYR
TLSSTLYRRP FNIGINNQQL SVLDGTEFAY GTSSNLPSAV YRKSGTVDSL DEIPPQNNNV
PPRQGFSHRL SHVSMFRSGF SNSSVSIIRA PMFSWIHRSA EFNNIIASDS ITQIPAVKGN
FLFNGSVISG PGFTGGDLVR LNSSGNNIQN RGYIEVPIHF PSTSTRYRVR VRYASVTPIH
LNVNWGNSSI FSNTVPATAT SLDNLQSSDF GYFESANAFT SSLGNIVGVR NFSGTAGVII
DRFEFIPVTA TLE

(SEQ ID NO:12) or a variant thereof having at least 90%, 95%, or more sequence identity to SEQ ID NO:12.

Another example of an insect resistant gene is a gene encoding a lectin. See, for example, Van Damme, et al., Plant Molec. Biol., 24:25 (1994), which discusses the nucleotide sequences of several Clivia miniata mannose-binding lectin genes. A vitamin-binding protein may also be used, such as avidin. See PCT application US93/06487 which describes the use of avidin and avidin homologues as larvicides against insect pests.

Yet another insect resistance gene is an enzyme inhibitor, for example, a protease or proteinase inhibitor or an amylase inhibitor. See, for example, Abe, et al., J. Biol. Chem., 262:16793 (1987) (nucleotide sequence of rice cysteine proteinase inhibitor), Huub, et al., Plant Molec. Biol., 21:985 (1993) (nucleotide sequence of cDNA encoding tobacco proteinase inhibitor I), and Sumitani et al., Biosci. Biotech. Biochem., 57:1243 (1993) (nucleotide sequence of Streptomyces nitrosporeus α-amylase inhibitor).

An insect-specific hormone or pheromone may also be used. See, for example, the disclosure by Hammock, et al., Nature, 344:458 (1990), of baculovirus expression of cloned juvenile hormone esterase, an inactivator of juvenile hormone; Gade, et al., Eds. Physiological System in Insects, Elsevier Academic Press, Burlington, Mass., (2007), describing allostatins and their potential use in pest control; and Palli, et al., Vitam. Horm., 73:59-100 (2005), disclosing use of ecdysteroid and ecdysteroid receptor in agriculture. The diuretic hormone receptor (DHR) was identified in Price, et al., Insect Mol. Biol., 13:469-480 (2004) as a candidate target of insecticides.

Still other examples include an insect-specific antibody or an immunotoxin derived therefrom and a developmental-arrestive protein. See Taylor, et al., Seventh Intl Symposium on Molecular Plant-Microbe Interactions, Edinburgh, Scotland, Abstract W97 (1994), which describes enzymatic inactivation in transgenic tobacco via production of single-chain antibody fragments. Numerous other examples of insect resistance have been described. See, for example, U.S. Pat. Nos. 6,809,078; 6,713,063; 6,686,452; 6,657,046; 6,645,497; 6,642,030; 6,639,054; 6,620,988; 6,593,293; 6,555,655; 6,538,109; 6,537,756; 6,521,442; 6,501,009; 6,468,523; 6,326,351; 6,313,378; 6,284,949; 6,281,016; 6,248,536; 6,242,241; 6,221,649; 6,177,615; 6,156,573; 6,153,814; 6,110,464; 6,093,695; 6,063,756; 6,063,597; 6,023,013; 5,959,091; 5,942,664; 5,942,658, 5,880,275; 5,763,245 and 5,763,241 all of which are incorporated by reference in their entirety.

C. Methods of Modulating Insect Resistance Using QTL-H

It has also been discovered that plants with QTL-H, alone, or in combination with other insect resistance imparting genes and QTLs including but not limited to the other insect resistance imparting genes and QTLs disclosed herein, exhibit increased insect resistance to leaf-chewing and some sucking insects. In particular, it has been discovered that QTL-H imparts resistance to sucking insects such as Megacopta cribraria, and plants that harbor QTL-H exhibit reduced insect infestation and increased crop yield compared to control plants without QTL-H. The resistance can be imparted by QTL-H in presence or absence of other insect resistance genes, constructs that modify gene product expression, and QTLs. Therefore, compositions and transgenic and hybrid plants including QTL-H, and methods of use thereof for reducing insect infection and increasing crop yield are also disclosed. In some embodiments, the plants include additional insect resistance genes, constructs that modify gene product expression, QTLs, or combinations thereof. In other embodiments, the plants do not include additional insect resistance genes, constructs that modify gene product expression, QTLs, or combinations thereof. For example, in some embodiments, the plant is resistant to insects without QTL-M or QTL-G.

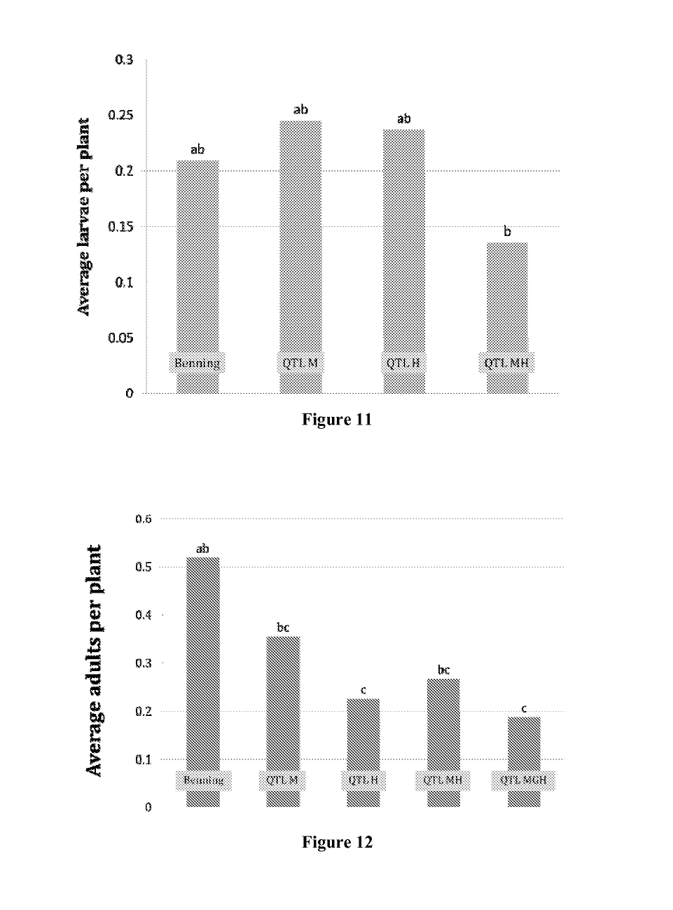

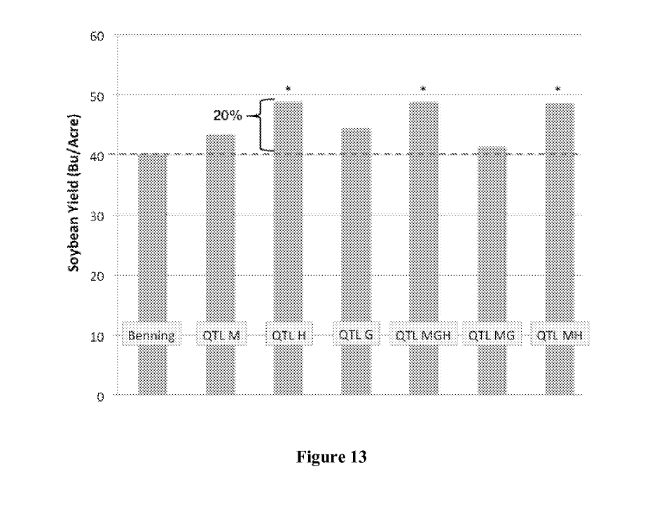

For example, the data presented in Example 8 shows that the QTL-H alone, or in combination with QTL-M, QTL-G, or a combination thereof can be effective to reduce feeding by kudzu bugs. FIG. 11 shows that nymph feeding is reduced on plants with a combination of QTL-H and QTL-M, and FIG. 12 shows that adult feeding is reduced on plants with QTL-H or QTL-M, or a combination thereof. FIG. 13 shows that crop yield is increased in plants with QTL-H alone, or in combination with QTL-M, QTL-G, or a combination thereof.

Therefore, transgenic and hybrid plants including QTL-H are disclosed. In some embodiments, the plants are used in method of increasing insect resistance against kudzu bugs. The plant can be a soybean plant. Preferably the plant is also a transgenic plant where the plant exhibits altered, reduced or inhibited expression or activity of the Glyma07g14530 flavonoid glucosyltransferase protein as discussed above. The plant can also contain QTL-M, QTL-G, or the combination thereof. The plants can be used to increase insect resistance or decrease feeding or infestation of larval and adult kudzu bugs, or increase crop yield.

### IV. Methods for Making Pest Resistant Plants

Non-naturally occurring plants employing one or more of the above disclosed compositions or methods of modulating pest resistance are also disclosed. In some embodiments the plants are transgenic plants, for example a transgenic plant expressing an antisense oligonucleotide that prevents, alters, reduces or inhibits expression of a flavonoid glucosyltransferase, such as Glyma07g14530, or a functional fragment, variant, ortholog or homolog thereof. In other embodiments, the transgenic plant expresses a gene of interest, such as an insect resistance gene, under the control of an herbivory inducible control sequence such as an expression control sequence of Glyma07g14530. In still other embodiments, the transgenic plants are characterized by a reduction or inhibition of a flavonoid glucosyltransferase, such as Glyma07g14530, and expression of one or more endogenous or heterologous insect resistance genes such as crylAc under the control of a Glyma07g14530 herbivory inducible control sequence.

The transgenic plant can include one or more insect resistance transgenes or insect resistant QTLs. For example, in some embodiments, the transgenic plant includes one or more alleles of a quantitative trait locus (QTL) including the Pb locus. The Pb locus conditions sharp (Pb) or blunt (pb) pubescence tip in soybean. The QTL for Pb imparts a sharp phenotype to the pubescence tip, which confers increased antixenosis and antibiosis to insects including Lepidopterans compared to a plant with blunt (pb) pubescence tip. Pb-containing QTL's and soybean accessions containing them are known in the art, see for example, Hulburt, et al., J. Econ. Entomol., 97(2):621-7 (2004), and Japanese accession, PI227687 respectively. Accordingly, in some embodiments the background of the transgenic plant is PI227687, or another plant having the Pb QTL. In some embodiments, the plant is an elite soybean cultivar introgressed with a least one allele of a Pb-containing QTL. Genetic marker(s) most closely associated with the Pb QTL include Sat_{—}112, Sat411 (Hulburt, D. J. 2002. Identifying additional insect resistance quantitative trait loci in soybean using simple sequence repeats. M.S. Thesis, University of Georgia, Athens. Warrington, C. V. 2006. Seed yield and insect resistance in near-isogenic soybean lines with introgressed resistance QTL from PI 229358. M.S. Thesis, University of Georgia, Athens).

In some embodiments, the transgenic plant includes one or more alleles of QTL-H. QTL-H is a soybean qualitative trait locus on chromosome 12 which confers antixenosis in PI229358 and PI171451 accessions with R² values ranging from 9 to 19% (Rector, et al., Crop Science, 9(2):531-538 (1999)). Accordingly, in some embodiments, the background of the transgenic plant is a plant having QTL-H. In other embodiments, the plant is an elite soybean cultivar introgressed with QTL-H. The genetic marker(s) most closely associated with QTL-H include Sat_{—}334, Satt541, Satt_{—}122, Sat_{—}118 (Parrott, et al., “Genomics of Insect-Soybean Interactions.” In: G. Stacey (ed.) Genetics and Genomics of Soybean. New York, N.Y.: Springer. p. 269-291(2008)).

In still other embodiments, the transgenic plant includes one or more alleles of QTL-G. QTL-G is a soybean qualitative trait locus on chromosome 18 which confers antibiosis (R²=19%) in PI229358 (Rector, et al., Crop Science, 40(1): 233-238 (2000)). Accordingly, the background of the transgenic plant can be a plant having QTL-G. The plant can be an elite soybean cultivar introgressed with QTL-G. The genetic marker(s) most closely associated with QTL-G include Satt472, Satt191 (Parrott, et al., “Genomics of Insect-Soybean Interactions.” In: G. Stacey (ed.) Genetics and Genomics of Soybean. New York, N.Y.: Springer. p. 269-291(2008)).

In still another embodiment, the insect-resistant plant contains one or more alleles from QTL-M in combination with one or more alleles from QTL-E (Pb). For example, a plant can be generated to contain both QTL-M and QTL-E. The plant can be transgenic or a hybrid.

The disclosed pest resistant plants typically have increased resistance to one or more plant eating animals, increased resistance to one or more plant eating insects, or combinations thereof relative to naturally occurring plants. The plant eating insects can have evolved mouthparts for chewing, snipping or tearing tissue, such as leaf-eating beetles or caterpillars; piercing-sucking insects, such as thrips and true-bugs, which use a tube-like mouthpart to syphon the liquid content of wounded cells; leafminer insects that develop in, and feed on, soft tissue between epidermal cell layers; insects such as aphids, whiteflies, and leafhoppers that are able to insert a specialized stylet between cells, then establishing a feeding site in the phloem; or combinations thereof. In a preferred embodiment, the transgenic plants have increased resistance to leaf chewing insects. The insect resistance can be antixenosis, antibiosis, tolerance or combinations thereof. In a preferred embodiment, the disclosed plants have an increased resistance to one or more insects including, but not limited to, lesser corn stalk borer, fall army worm, cut worm, coleopteran such as the Mexican bean beetle (MBB), several soybean lepidopteran pests such as soybean looper (SBL), velvetbean caterpillar (VBC), beet armyworm (BAW), corn earworm (CEW), tobacco budworm (TBW), and bean plataspid (i.e., Megacopta cribraria (kudzu bug)).

A. Constructs and Vectors

1. Recombinant Expression

Vectors and constructs containing a flavonoid glucosyltransferase gene or coding sequence, such as Glyma07g14530, or a fragment, variant, ortholog or homolog thereof can be operably linked to an endogenous or heterologous expression control sequence are also disclosed. The constructs can include an expression cassette containing an Glyma07g14530 gene or coding sequence, for example SEQ ID NO: 1, 2, 3, 4, 5, or 6, or a nucleic acid encoding the amino acid sequence of SEQ ID NO:7, 8, or 9.

2. Antisense

Antisense oligonucleotides that target a flavonoid glucosyltransferase such as Glyma07g14530, or and ortholog or a homolog thereof are also disclosed. Antisense oligonucleotides include, but are not limited to, RNAi, dsRNA, miRNA, siRNA, or transacting small-interfering RNAs (tasiRNA) that target the flavonoid glucosyltransferase mRNA in a plant, and delay, inhibit, or prevent expression of the flavonoid glucosyltransferase gene or gene product in plants. Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication.

Antisense molecules can be designed based on the sequence of the target molecule, for example Glyma07g14530 coding sequences including, but not limited to, SEQ ID NO: 1, 2, 3, 4, 5, or 6, or a nucleic acid encoding the amino acid sequence of SEQ ID NO:7, 8, or 9. Methods of designing antisense molecules directed to a target sequence, for example SEQ ID NO: 1, 2, 3, 4, 5, or 6, or a nucleic acid encoding the amino acid sequence of SEQ ID NO:7, 8, or 9 are well also well known in the art. See for example, Elbashir, et al., Methods, 26:199-213 (2002).

The production of siRNA from a vector is more commonly done through the transcription of a short hairpin RNAs (shRNAs). Accordingly, vectors and constructs containing a nucleic acid sequence that silences Glyma07g14530 gene expression (e.g., siRNA, RNAi, shRNA, tasiRNA) operably linked to a heterologous expression control sequence are also disclosed.

3. Genes of Interest

Methods of modifying a plant gene, polynucleotide, or coding sequence to be pest resistant are also disclosed. The method generally involves operably linking a Glyma07g14530 herbivory inducible control sequence to a polynucleotide of interest. The polynucleotide of interest can be a coding sequence, for example a sequence encoding a polypeptide (with or without introns), or non-coding sequence such as an antisense or inhibitory nucleic acid. In some embodiments the polynucleotide includes a cDNA of a polypeptide of interest, for example an insect resistance gene.

4. Transformation Constructs

Transformation constructs including the disclosed nucleic acids are also disclosed. Constructs can be engineered such that transformation of the nuclear genome and expression of transgenes from the nuclear genome occurs. Alternatively, transformation constructs can be engineered such that transformation of the plastid genome and expression of the plastid genome occurs. Transformation constructs can be used, for example, to express an antisense oligonucleotide that reduces or silences gene expression of a flavonoid glucosyltransferase gene or coding sequence, such as Glyma07g14530, or introduce nucleic acids endogenous or heterologous insect resistant polypeptides operably linked to an herbivory inducible promoter.

Generally, the nucleic acid sequences disclosed are operably linked to a suitable promoter expressible in plants, and used to modulate insect resistance in a plant. Expression cassettes containing the disclosed nucleic acids may also include any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes can then be easily transferred to the plant transformation vectors. Representative plant transformation vectors are described in plant transformation vector options available (Gene Transfer to Plants (1995), Potrykus, et al., G. eds. Springer-Verlag Berlin Heidelberg New York; “Transgenic Plants: A Production System for Industrial and Pharmaceutical Proteins” (1996), Owen, M. R. L. and Pen, J. eds. John Wiley & Sons Ltd. England and Methods in Plant Molecular biology—a laboratory course manual (1995), Maliga, P., Klessig, D. F., Cashmore, A. R., Gruissem, W. and Varner, J. E. eds. Cold Spring Laboratory Press, New York).

An additional approach is to use a vector to specifically transform the plant plastid chromosome by homologous recombination (U.S. Pat. No. 5,545,818 to McBride, et al.), in which case it is possible to take advantage of the prokaryotic nature of the plastid genome and insert a number of transgenes as an operon.

The following is a description of various components of typical expression cassettes.

5. Promoters

Plant promoters can be selected to control the expression of the transgene in different plant tissues or organelles, for all of which methods are known to those skilled in the art (Gasser, et al., Science, 244:1293-99 (1989)). In a preferred embodiment, promoters are selected from those of plant or prokaryotic origin that are known to yield high expression in plastids. In certain embodiments the promoters are inducible. Inducible plant promoters are known in the art.

The transgenes can be inserted into an existing transcription unit (such as, but not limited to, psbA) to generate an operon. However, other insertion sites can be used to add additional expression units as well, such as existing transcription units and existing operons (e.g., atpE, accD). Such methods are described in, for example, U.S. Pat. App. Pub. 2004/0137631, which is incorporated herein by reference in its entirety. For an overview of other insertion sites used for integration of transgenes into the tobacco plastome, see Staub (Staub, J. M., “Expression of Recombinant Proteins via the Plastid Genome,” in: Vinci V A, Parekh S R (eds.) Handbook of Industrial Cell Culture: Mammalian, and Plant Cells, pp. 259-278, Humana Press Inc., Totowa, N.J. (2002)).

In general, the promoter can be from any class I, II or III gene. For example, any of the following plastidial promoters and/or transcription regulation elements can be used for expression in plastids. Sequences can be derived from the same species as that used for transformation. Alternatively, sequences can be derived from other species to decrease homology and to prevent homologous recombination with endogenous sequences.

For instance, the following plastidial promoters can be used for expression in plastids.

PrbcL promoter (Allison, et al., EMBO J. 15:2802-2809 (1996); Shiina, et al., Plant Cell 10:1713-1722 (1998));

PpsbA promoter (Agrawal, et al., Nucleic Acids Research 29:1835-1843 (2001));

Prrn 16 promoter (Svab, et al., Proc. Natl. Acad. Sci. USA 90:913-917 (1993); Allison, et al., EMBO J. 15:2802-2809 (1996));

PaccD promoter (Hajdukiewicz P T J, Allison L A, Maliga P, EMBO J. 16:4041-4048 (1997); WO 97/06250);

PclpP promoter (Hajdukiewicz, et al., EMBO J. 16:4041-4048 (1997); WO 99/46394);

PatpB, PatpI, PpsbB promoters (Hajdukiewicz, et al., EMBO J. 16:4041-4048 (1997));

PrpoB promoter (Liere K, Maliga P, EMBO J. 18:249-257 (1999);

PatpB/E promoter (Kapoor, et al., Plant J. 11:327-337 (1997)).

In addition, prokaryotic promoters (such as those from, e.g., E. coli or Synechocystis) or synthetic promoters can also be used.

Promoters vary in their strength, i.e., ability to promote transcription. Depending upon the host cell system utilized, any one of a number of suitable promoters known in the art may be used. For example, for constitutive expression, the CaMV 35S promoter, the rice actin promoter, or the ubiquitin promoter may be used. For example, for regulatable expression, the chemically inducible PR-1 promoter from tobacco or Arabidopsis may be used (see, e.g., U.S. Pat. No. 5,689,044 to Ryals, et al.).

A suitable category of promoters is that which is wound inducible. Numerous promoters have been described which are expressed at wound sites. Preferred promoters of this kind include those described by Stanford, et al., Mol. Gen. Genet. 215: 200-208 (1989), Xu et al. Plant Molec. Biol. 22:573-588 (1993), Logemann, et al., Plant Cell 1:151-158 (1989), Rohrmeier, et al., Plant Molec. Biol. 22:783-792 (1993), Firek, et al., Plant Molec. Biol. 22:129-142 (1993), and Warner, et al., Plant J., 3:191-201 (1993).

Suitable tissue specific expression patterns include green tissue specific, root specific, stem specific, and flower specific. Promoters suitable for expression in green tissue include many which regulate genes involved in photosynthesis, and many of these have been cloned from both monocotyledons and dicotyledons. A suitable promoter is the maize PEPC promoter from the phosphoenol carboxylase gene (Hudspeth, et al., Plant Molec. Biol. 12:579-589 (1989)). A suitable promoter for root specific expression is that described by de Framond, FEBS, 290: 103-106 (1991); EP 0 452 269 to de Framond and a root-specific promoter is that from the T-1 gene. A suitable stem specific promoter is that described in U.S. Pat. No. 5,625,136 and which drives expression of the maize trpA gene.

The promoter can be a relatively weak plant expressible promoter. Thus, the promoter can in some embodiments initiate and control transcription of the operably linked nucleic acids about 10 to about 100 times less efficient that an optimal CaMV35S promoter. Relatively weak plant expressible promoters include the promoters or promoter regions from the opine synthase genes of Agrobacterium spp. such as the promoter or promoter region of the nopaline synthase, the promoter or promoter region of the octopine synthase, the promoter or promoter region of the mannopine synthase, the promoter or promoter region of the agropine synthase and any plant expressible promoter with comparably activity in transcription initiation. Other relatively weak plant expressible promoters may be dehiscence zone selective promoters, or promoters expressed predominantly or selectively in dehiscence zone and/or valve margins of fruits, such as the promoters described in WO97/13865.

Cis-regulatory elements from the promoter of photoperiod-responsive genes, coordinated motifs integrating hormones and stresses to photoperiod responses, and the promoters of photo-responsive genes such as those described in Mongkolsiriwatana, Katsetsart J. (Nat. Sci) 43: 164-177 (2009), can also be used.

6. Transcriptional Terminators

A variety of transcriptional terminators are available for use in expression cassettes. These are responsible for the termination of transcription beyond the transgene and its correct polyadenylation. Appropriate transcriptional terminators are those that are known to function in plants and include the CaMV 35S terminator, the tml terminator, the nopaline synthase terminator and the pea rbcS E9 terminator. These are used in both monocotyledonous and dicotyledonous plants.

At the extreme 3′ end of the transcript, a polyadenylation signal can be engineered. A polyadenylation signal refers to any sequence that can result in polyadenylation of the mRNA in the nucleus prior to export of the mRNA to the cytosol, such as the 3′ region of nopaline synthase (Bevan, et al., Nucleic Acids Res., 11:369-385 (1983)).

7. Sequences for Expression Enhancement or Regulation

Numerous sequences have been found to enhance gene expression from within the transcriptional unit and these sequences can be used in conjunction with the genes to increase their expression in transgenic plants. For example, various intron sequences such as introns of the maize Adhl gene have been shown to enhance expression, particularly in monocotyledonous cells. In addition, a number of non-translated leader sequences derived from viruses are also known to enhance expression, and these are particularly effective in dicotyledonous cells.

8. Coding Sequence Optimization

The coding sequence of the disclosed genes can be genetically engineered by altering the coding sequence for optimal expression (also referred to herein as “codon optimized”) in the crop species of interest. Methods for modifying coding sequences to achieve optimal expression in a particular crop species are well known (see, e.g. Perlak, et al., Proc. Natl. Acad. Sci. USA 88: 3324 (1991); and Koziel, et al, Biotechnol. 11: 194 (1993)). Therefore, in some embodiments, the disclosed nucleic acids sequences, or fragments or variants thereof, are genetically engineered for optimal expression in the crop species of interest.

9. Selectable Markers

Genetic constructs may encode a selectable marker to enable selection of plastid transformation events. There are many methods that have been described for the selection of transformed plants [for review see (Miki et al., Journal of Biotechnology, 107:193-232 (2004) and references incorporated within]. Selectable marker genes that have been used extensively in plants include the neomycin phosphotransferase gene nptII (U.S. Pat. No. 5,034,322, U.S. Pat. No. 5,530,196), hygromycin resistance gene (U.S. Pat. No. 5,668,298), the bar gene encoding resistance to phosphinothricin (U.S. Pat. No. 5,276,268), the expression of aminoglycoside 3″-adenyltransferase (aadA) to confer spectinomycin resistance (U.S. Pat. No. 5,073,675), the use of inhibition resistant 5-enolpyruvyl-3-phosphoshikimate synthetase (U.S. Pat. No. 4,535,060) and methods for producing glyphosate tolerant plants (U.S. Pat. No. 5,463,175; U.S. Pat. No. 7,045,684). Methods of plant selection that do not use antibiotics or herbicides as a selective agent have been previously described and include expression of glucosamine-6-phosphate deaminase to inactive glucosamine in plant selection medium (U.S. Pat. No. 6,444,878) and a positive/negative system that utilizes D-amino acids (Erikson, et al., Nat Biotechnol, 22:455-8 (2004). European Patent Publication No. EP 0 530 129 A1 describes a positive selection system which enables the transformed plants to outgrow the non-transformed lines by expressing a transgene encoding an enzyme that activates an inactive compound added to the growth media. U.S. Pat. No. 5,767,378 describes the use of mannose or xylose for the positive selection of transgenic plants. Methods for positive selection using sorbitol dehydrogenase to convert sorbitol to fructose for plant growth have also been described (WO 2010/102293). Screenable marker genes include the beta-glucuronidase gene (Jefferson, et al., EMBO J. 6:3901-3907 (1987); U.S. Pat. No. 5,268,463) and native or modified green fluorescent protein gene (Cubitt, et al., Trends Biochem. Sci. 20:448-455 (1995); Pan, et al., Plant Physiol. 112:893-900 (1996)).

Transformation events can also be selected through visualization of fluorescent proteins such as the fluorescent proteins from the nonbioluminescent Anthozoa species which include DsRed, a red fluorescent protein from the Discosoma genus of coral (Matz, et al., Nat Biotechnol, 17:969-73 (1999)). An improved version of the DsRed protein has been developed (Bevis, et al., Nat Biotech, 20:83-87 (2002)) for reducing aggregation of the protein. Visual selection can also be performed with the yellow fluorescent proteins (YFP) including the variant with accelerated maturation of the signal (Nagai, et al., Nat Biotech, 20:87-90 (2002), the blue fluorescent protein, the cyan fluorescent protein, and the green fluorescent protein (Sheen, et al., Plant J, 8:777-84 (1995); Davis, et al., Plant Molecular Biology, 36:521-528 (1998)). A summary of fluorescent proteins can be found in Tzfira, et al., Plant Molecular Biology, 57:503-516 (2005) and Verkhusha, et al., Nat Biotech, 22:289-296 (2004) whose references are incorporated in entirety. Improved versions of many of the fluorescent proteins have been made for various applications. Use of the improved versions of these proteins or the use of combinations of these proteins for selection of transformants will be obvious to those skilled in the art. It is also practical to simply analyze progeny from transformation events for the presence of the PHB thereby avoiding the use of any selectable marker.

For plastid transformation constructs, a preferred selectable marker is the spectinomycin-resistant allele of the plastid 16S ribosomal RNA gene (Staub, et al., Plant Cell 4:39-45 (1992); Svab, et al., Proc. Natl. Acad. Sci. USA 87:8526-8530 (1990)). Selectable markers that have since been successfully used in plastid transformation include the bacterial aadA gene that encodes aminoglycoside 3′-adenyltransferase (AadA) conferring spectinomycin and streptomycin resistance (Svab, et al., Proc. Natl. Acad. Sci. USA, 90:913-917 (1993), nptII that encodes aminoglycoside phosphotransferase for selection on kanamycin (Carrer, et al., Mol. Gen. Genet. 241:49-56 (1993); Lutz, et al., Plant J., 37: 906-913 (2004); Lutz K A, et al., Plant Physiol. 145: 1201-1210 (2007)), aphA6, another aminoglycoside phosphotransferase (Huang F-C, et al, Mol. Genet. Genomics 268: 19-27 (2002)), and chloramphenicol acetyltransferase (Li, et al., Plant Mol Biol, DOI 10.1007/s11103-010-9678-4 (2010)). Another selection scheme has been reported that uses a chimeric betaine aldehyde dehydrogenase gene (BADH) capable of converting toxic betaine aldehyde to nontoxic glycine betaine (Daniell, et al., Curr. Genet., 39:109-116 (2001)).

10. Targeting Sequences

The disclosed vectors and constructs may further include, within the region that encodes the protein to be expressed, one or more nucleotide sequences encoding a targeting sequence. A “targeting” sequence is a nucleotide sequence that encodes an amino acid sequence or motif that directs the encoded protein to a particular cellular compartment, resulting in localization or compartmentalization of the protein. Presence of a targeting amino acid sequence in a protein typically results in translocation of all or part of the targeted protein across an organelle membrane and into the organelle interior. Alternatively, the targeting peptide may direct the targeted protein to remain embedded in the organelle membrane. The “targeting” sequence or region of a targeted protein may contain a string of contiguous amino acids or a group of noncontiguous amino acids. The targeting sequence can be selected to direct the targeted protein to a plant organelle such as a nucleus, a microbody (e.g., a peroxisome, or a specialized version thereof, such as a glyoxysome) an endoplasmic reticulum, an endosome, a vacuole, a plasma membrane, a cell wall, a mitochondria, a chloroplast or a plastid. A chloroplast targeting sequence is any peptide sequence that can target a protein to the chloroplasts or plastids, such as the transit peptide of the small subunit of the alfalfa ribulose-biphosphate carboxylase (Khoudi, et al., Gene, 197:343-351 (1997)). A peroxisomal targeting sequence refers to any peptide sequence, either N-terminal, internal, or C-terminal, that can target a protein to the peroxisomes, such as the plant C-terminal targeting tripeptide SKL (Banjoko, et al., Plant Physiol., 107:1201-1208 (1995); Wallace et al., Plant Organellular Targeting Sequences, in Plant Molecular Biology, Ed. R. Croy, BIOS Scientific Publishers Limited 287-288 (1993), and peroxisomal targeting in plant is shown in M. Volokita, The Plant J., 361-366 (1991)).

Plastid targeting sequences are known in the art and include the chloroplast small subunit of ribulose-1,5-bisphosphate carboxylase (Rubisco) (de Castro Silva Filho et al., Plant Mol. Biol. 30:769-780 (1996); Schnell, et al. J. Biol. Chem. 266(5):3335-3342 (1991)); 5-(enolpyruvyl)shikimate-3-phosphate synthase (EPSPS) (Archer, et al., J. Bioenerg. Biomemb. 22(6):789-810 (1990)); tryptophan synthase (Zhao, et al., J. Biol. Chem. 270(11):6081-6087 (1995)); plastocyanin (Lawrence, et al., J. Biol. Chem. 272(33):20357-20363 (1997)); chorismate synthase (Schmidt, et al., J. Biol. Chem. 268(36):27447-27457 (1993)); and the light harvesting chlorophyll a/b binding protein (LHBP) (Lamppa, et al, J. Biol. Chem. 263:14996-14999 (1988)). See also Von Heijne, et al., Plant Mol. Biol. Rep. 9:104-126 (1991); Clark, et al., J. Biol. Chem. 264:17544-17550 (1989); Della-Cioppa, et al., Plant Physiol. 84:965-968 (1987); Romer, et al., Biochem. Biophys. Res. Commun. 196:1414-1421 (1993); and Shah, et al., Science 233:478-481 (1986). Alternative plastid targeting signals have also been described in the following: US 2008/0263728; Miras, et al., J Biol Chem, 277:49 (2002): 47770-8; Miras, et al., J Biol Chem, 282:29482-29492 (2007).

11. Plants and Tissues for Transfection, Introgression, and Breeding

Both dicotyledons (“dicots”) and monocotyledons (“monocots”) can be used in the disclosed positive selection system. Monocot seedlings typically have one cotyledon (seed-leaf), in contrast to the two cotyledons typical of dicots. Eudicots are dicots whose pollen has three apertures (i.e. triaperturate pollen), through one of which the pollen tube emerges during pollination. Eudicots contrast with the so-called ‘primitive’ dicots, such as the magnolia family, which have uniaperturate pollen (i.e. with a single aperture).

Monocots include one of the large divisions of Angiosperm plants (flowering plants with seeds protected within a vessel). They are herbaceous plants with parallel veined leaves and have an embryo with a single cotyledon, as opposed to dicot plants (dicotyledonous), which have an embryo with two cotyledons. Most of the important staple crops of the world, the so-called cereals, such as wheat, barley, rice, maize, sorghum, oats, rye and millet, are monocots. Thus, the plant can be a grass, such as wheat, barley, rice, maize, sorghum, oats, rye and millet.

The plant can therefore be a cereal crop such as wheat, oat, barley, or rice; a forage such as bahiagrass, dallisgrass, kleingrass, guineagrass, reed canarygrass, orchardgrass, ricegrass, foxtail, or vetch; a legume such as soybean, lentil, or chickpea; an oilseed such as canola; a vegetable such as onion or carrot; or a specialty crop such as caraway, hemp, or sesame.

In some embodiments, the plant is a soybean. For example, the soybean plant can be Jack, Resnik, Williams 82, Corsoy, Crawford, Hutcheson, Kunitz, Champ, Benning, or Woodruff. Additional suitable soybean varieties are available from both academic and commercial institutions, such as—for example—the University of Guelph (Ontario Agricultural College; e.g. soybean varieties RCAT Staples, Westag 97, RCAT Bobcat, OAC Prudence, OAC Woodstock, OAC 9908), or soybean varieties from Daryland or Soygenetics. Additional suitable varieties are P1548402 (Peking), P1437654 (Er-hejjan), P1438489 (Chiquita), P1507354 (Tokei 421), P1548655 (Forrest), P1548988 (Pickett), P188788, P1404198 (Sun Huan Do), P1404166 (Krasnoaarmejkaja), Hartwig, Manokin, Doles, Dyer, and Custer.

In some embodiments, the plant is a miscanthus. Thus, the plant can be of the species Miscanthus floridulus, Miscanthus x. giganteus, Miscanthus sacchariflorus (Amur silver-grass), Miscanthus sinensis, Miscanthus tinctorius, or Miscanthus transmorrisonensis.

Additional representative plants useful in the compositions and methods disclosed herein include the Brassica family including sp. napus, rapa, oleracea, nigra, carinata and juncea; industrial oilseeds such as Camelina sativa, Crambe, Jatropha, castor; Arabidopsis thaliana; soybean; cottonseed; sunflower; palm; coconut; rice; safflower; peanut; mustards including Sinapis alba; sugarcane and flax.

Crops harvested as biomass, such as silage corn, alfalfa, switchgrass, or tobacco, also are useful with the methods disclosed herein.

Representative tissues for transformation using these vectors include protoplasts, cells, callus tissue, leaf discs, pollen, and meristems.

B. Plant Transformation Techniques

The transformation of suitable agronomic plant hosts using vectors expressing transgenes can be accomplished with a variety of methods and plant tissues. Representative transformation procedures include Agrobacterium-mediated transformation, biolistics, microinjection, electroporation, polyethylene glycol-mediated protoplast transformation, liposome-mediated transformation, and silicon fiber-mediated transformation (U.S. Pat. No. 5,464,765 to Coffee, et al.; “Gene Transfer to Plants” (Potrykus, et al., eds.) Springer-Verlag Berlin Heidelberg New York (1995); “Transgenic Plants: A Production System for Industrial and Pharmaceutical Proteins” (Owen, et al., eds.) John Wiley & Sons Ltd. England (1996); and “Methods in Plant Molecular Biology: A Laboratory Course Manual” (Maliga et al. eds.) Cold Spring Laboratory Press, New York (1995)).

Plants can be transformed by a number of reported procedures (U.S. Pat. No. 5,015,580 to Christou, et al.; U.S. Pat. No. 5,015,944 to Bubash; U.S. Pat. No. 5,024,944 to Collins, et al.; U.S. Pat. No. 5,322,783 to Tomes et al.; U.S. Pat. No. 5,416,011 to Hinchee et al.; U.S. Pat. No. 5,169,770 to Chee et al.). A number of transformation procedures have been reported for the production of transgenic maize plants including pollen transformation (U.S. Pat. No. 5,629,183 to Saunders et al.), silicon fiber-mediated transformation (U.S. Pat. No. 5,464,765 to Coffee et al.), electroporation of protoplasts (U.S. Pat. No. 5,231,019 Paszkowski et al.; U.S. Pat. No. 5,472,869 to Krzyzek et al.; U.S. Pat. No. 5,384,253 to Krzyzek et al.), gene gun (U.S. Pat. No. 5,538,877 to Lundquist et al. and U.S. Pat. No. 5,538,880 to Lundquist et al.), and Agrobacterium-mediated transformation (EP 0 604 662 A1 and WO 94/00977 both to Hiei Yukou et al.). The Agrobacterium-mediated procedure is particularly preferred as single integration events of the transgene constructs are more readily obtained using this procedure which greatly facilitates subsequent plant breeding. Plants can be transformed by particle bombardment (U.S. Pat. No. 5,004,863 to Umbeck and U.S. Pat. No. 5,159,135 to Umbeck). Sunflower can be transformed using a combination of particle bombardment and Agrobacterium infection (EP 0 486 233 A2 to Bidney, Dennis; U.S. Pat. No. 5,030,572 to Power et al.). Flax can be transformed by either particle bombardment or Agrobacterium-mediated transformation. Switchgrass can be transformed using either biolistic or Agrobacterium mediated methods (Richards, et al., Plant Cell Rep., 20:48-54 (2001); Somleva, et al., Crop Science, 42:2080-2087 (2002)). Methods for sugarcane transformation have also been described (Franks, et al., Aust. J. Plant Physiol. 18:471-480 (1991); WO 2002/037951 to Elliott, et al., et al.).

Recombinase technologies which are useful in practicing the current invention include the cre-lox, FLP/FRT and Gin systems. Methods by which these technologies can be used for the purpose described herein are described for example in (U.S. Pat. No. 5,527,695 to Hodges et al.; Dale, et al., Proc. Natl. Acad. Sci. USA, 88:10558-10562 (1991); Medberry, et al., Nucleic Acids Res., 23: 485-490 (1995)).

Engineered minichromosomes can also be used to express one or more genes in plant cells. Cloned telomeric repeats introduced into cells may truncate the distal portion of a chromosome by the formation of a new telomere at the integration site. Using this method, a vector for gene transfer can be prepared by trimming off the arms of a natural plant chromosome and adding an insertion site for large inserts (Yu, et al., Proc Natl Acad Sci USA, 103:17331-6 (2006); Yu, et al., Proc Natl Acad Sci USA,104:8924-9 (2007)). The utility of engineered minichromosome platforms has been shown using Cre/lox and FRT/FLP site-specific recombination systems on a maize minichromosome where the ability to undergo recombination was demonstrated (Yu, et al., Proc Natl Acad Sci USA, 103:17331-6 (2006); Yu, et al., Proc Natl Acad Sci USA,104:8924-9 (2007)). Such technologies could be applied to minichromosomes, for example, to add genes to an engineered plant. Site specific recombination systems have also been demonstrated to be valuable tools for marker gene removal (Kerbach, et al., Theor. Appl. Genet. 111:1608-1616 (2005)), gene targeting Chawla, et al., Plant Biotechnol. J, 4:209-218 (2006); Choi, et al., Nucleic Acids Res., 28: E19 (2000); Srivastava, et al., Plant Mol Biol. 46:561-566 (2001); Lyznik, et al., Nucleic Acids Res., 21:969-975 (1993)) and gene conversion (Djukanovic, et al., Plant Biotechnol J., 4:345-357 (2006)).

An alternative approach to chromosome engineering in plants involves in vivo assembly of autonomous plant minichromosomes (Carlson, et al., PLoS Genet., 3:1965-74 (2007)). Plant cells can be transformed with centromeric sequences and screened for plants that have assembled autonomous chromosomes de novo. Useful constructs combine a selectable marker gene with genomic DNA fragments containing centromeric satellite and retroelement sequences and/or other repeats.

Another approach useful to the described invention is Engineered Trait Loci (“ETL”) technology (U.S. Pat. No. 6,077,697; US Patent Application 2006/0143732). This system targets DNA to a heterochromatic region of plant chromosomes, such as the pericentric heterochromatin, in the short arm of acrocentric chromosomes. Targeting sequences may include ribosomal DNA (rDNA) or lambda phage DNA. The pericentric rDNA region supports stable insertion, low recombination, and high levels of gene expression. This technology is also useful for stacking of multiple traits in a plant (US Patent Application 2006/0246586).

Zinc-finger nucleases (ZFNs) are also useful for practicing the invention in that they allow double strand DNA cleavage at specific sites in plant chromosomes such that targeted gene insertion or deletion can be performed (Shukla, et al., Nature, 459(7245):437-41 (2009)); Townsend et al., Nature, 459(7245):442-5 (2009)).

Following transformation by any one of the methods described above, the following procedures can, for example, be used to obtain a transformed plant expressing the transgenes: select the plant cells that have been transformed on a selective medium, regenerate the plant cells that have been transformed to produce differentiated plants, select transformed plants expressing the transgene producing the desired level of desired polypeptide(s) in the desired tissue and cellular location.

Transformation techniques for dicotyledons are well known in the art and include Agrobacterium-based techniques and techniques that do not require Agrobacterium. Non-Agrobacterium techniques involve the uptake of heterologous genetic material directly by protoplasts or cells. This is accomplished by PEG or electroporation mediated uptake, particle bombardment-mediated delivery, or microinjection. In each case the transformed cells may be regenerated to whole plants using standard techniques known in the art.

Transformation of most monocotyledon species has now become somewhat routine. Preferred techniques include direct gene transfer into protoplasts using PEG or electroporation techniques, particle bombardment into callus tissue or organized structures, as well as Agrobacterium-mediated transformation.

Plants from transformation events are grown, propagated and bred to yield progeny with the desired trait, and seeds are obtained with the desired trait, using processes well known in the art.

C. Plastid Transformation

In another embodiment the transgene is directly transformed into the plastid genome. Plastid transformation technology is extensively described in U.S. Pat. No. 5,451,513 to Maliga et al., U.S. Pat. No. 5,545,817 to McBride et al., and U.S. Pat. No. 5,545,818 to McBride et al., in PCT application no. WO 95/16783 to McBride et al., and in McBride, et al., Proc. Natl. Acad. Sci. USA 91:7301-7305 (1994). The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Suitable plastids that can be transfected include, but are not limited to, chloroplasts, etioplasts, chromoplasts, leucoplasts, amyloplasts, proplastids, statoliths, elaioplasts, proteinoplasts and combinations thereof.

D. Methods for Reproducing Transgenic Plants

Following transformation by any one of the methods described above, the following procedures can be used to obtain a transformed plant expressing the transgenes: select the plant cells that have been transformed on a selective medium; regenerate the plant cells that have been transformed to produce differentiated plants; select transformed plants expressing the transgene producing the desired level of desired polypeptide(s) in the desired tissue and cellular location.

In plastid transformation procedures, further rounds of regeneration of plants from explants of a transformed plant or tissue can be performed to increase the number of transgenic plastids such that the transformed plant reaches a state of homoplasmy (all plastids contain uniform plastomes containing transgene insert).

The cells that have been transformed may be grown into plants in accordance with conventional techniques. See, for example, McCormick et al. Plant Cell Reports, 5:81-84(1986). These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved.

In some scenarios, it may be advantageous to insert a multi-gene pathway into the plant by crossing of lines containing portions of the pathway to produce hybrid plants in which the entire pathway has been reconstructed. This is especially the case when high levels of product in a seed compromises the ability of the seed to germinate or the resulting seedling to survive under normal soil growth conditions. Hybrid lines can be created by crossing a line containing one or more the transgene miRNA targeting sequence constructs disclosed herein with a line containing the miRNA. Use of lines that possess cytoplasmic male sterility (Esser, et al., Progress in Botany, Springer Berlin Heidelberg. 67:31-52 (2006)) with the appropriate maintainer and restorer lines allows these hybrid lines to be produced efficiently. Cytoplasmic male sterility systems are already available for some Brassicaceae species (Esser, et al., Progress in Botany, Springer Berlin Heidelberg. 67:31-52 (2006)).

E. Breeding

1. Methods of Breeding Hybrid Plants

Field crops are bred through techniques that take advantage of the plant's method of pollination. A plant is self-pollinated if pollen from one flower is transferred to the same or another flower of the same plant. A plant is cross-pollinated if the pollen comes from a flower on a different plant. Plants that have been self-pollinated and selected for type for many generations become homozygous at almost all gene loci and produce a uniform population of true breeding progeny. A cross between two different homozygous lines produces a uniform population of hybrid plants that may be heterozygous for many gene loci. A cross of two plants each heterozygous at a number of gene loci will produce a population of hybrid plants that differ genetically and may not be uniform.

The plants disclosed herein include hybrid plants which can be produced using any known breeding techniques. Hybrids are the product of a cross between genetically different parents. The development of hybrids in a plant breeding program often involves the development of homozygous inbred lines, the crossing of these lines, and the evaluation of the crosses. Most plant breeding programs combine the genetic backgrounds from two or more inbred lines or various other broad-based sources into breeding pools from which new inbred lines are developed by selfing and selection of desired phenotypes. Hybrids can also be used as a source of plant breeding material or as source populations from which to develop or derive new plant lines. The expression of a trait in a hybrid may exceed the midpoint of the amount expressed by the two parents, which is known as hybrid vigor.

Plant breeding techniques known in the art include, but are not limited to, recurrent selection, pedigree breeding, DNA marker enhanced selection, genetic marker enhanced selection and transformation. Inbred lines may, for instance, be derived from hybrids by using said methods as pedigree breeding and recurrent selection breeding. Newly developed inbreds are crossed with other inbred lines and the hybrids from these crosses are evaluated to determine which of those have commercial potential.

Pedigree breeding is a system of breeding in which individual plants are selected in the segregating generations from a cross on the basis of their desirability judged individually and on the basis of a pedigree record.

Recurrent selection is a breeding method based upon intercrossing selected individuals followed by continuing cycles of selection and intercrossing to increase the frequency of desired alleles in the population.

Recurrent selection may, for instance, be performed by backcross breeding, which involves a system of breeding whereby recurrent backcrosses are made to one of the parents of a hybrid, accompanied by selection for a specific character or characters. The backcross is the cross of a hybrid to either of its parents. Backcrossing can for instance be used to transfer a specific desirable trait that is present in a donor plant line to another, superior plant line (e.g. an inbred line) that lacks that trait.

The first step of this process involves crossing the superior plant line (recurrent parent) to a donor plant line (non-recurrent parent), that carries the appropriate gene(s) for the trait in question. The progeny of this cross is then mated back to the superior recurrent parent followed by selection in the resultant progeny for the desired trait to be transferred from the non-recurrent parent. After five or more backcross generations with selection for the desired trait and for the germplasm inherited from the recurrent parent, the progeny will be homozygous for loci controlling the characteristic being transferred, but will be like the superior parent for essentially all other genes. The last backcross generation is then selfed to give pure breeding progeny for the gene(s) being transferred. A hybrid developed from inbreds containing the transferred gene(s) is essentially the same as a hybrid developed from the same inbreds without the transferred gene(s).

Introgression, also known as introgressive hybridization, is another hybrid breeding technique. Introgressive hybridization results in the movement of one or more genes (gene flow) from one species into the gene pool of another by repeated backcrossing of an interspecific hybrid with one of its parent species. Introgression is a long-term process; it may take many hybrid generations before the backcrossing is completed.

One example of introgressive hybridization is known as advanced backcross-self breeding. The AB method consists of crossing one parental line (donor parent) with another parental line (recurrent parent) to produce F₁ progeny. The F₁ progeny can be optionally self-crossed to generate F₂ progeny. The F₁ or F₂ progeny is then crossed with the recurrent parent to produce a backcross progeny (BC₁). BC₁ are selected and crossed again with the recurrent parent resulting in a second generation of backcross progeny (BC₂). BC₂ can be optionally backcrossed with the recurrent parent to generate a third generation of backcross progeny (BC₃). Plants from the BC₂ and/or the BC₃ generation are then allowed to self-pollinate for one or more generations, followed by evaluation for presence of the characteristics transferred from the donor parent. Methods for evaluating the presence of donor characteristics can be accomplished using any technique known in the art. Specific methods for evaluating the presence of donor characteristics, are described in detail in the examples below.

The disclosed QTLs, transgenes, or combinations thereof can be used to establish a breeding program to cultivate hybrid plants with one or more of desired phenotypic characteristics. Because quantitative traits are phenotypic characteristics that vary in degree and may include environmental influence, breeders may also take into consideration the breeding environment and or breeding location when cultivating the plants disclosed herein.

2. Genotyping Hybrid Plants

Quantitative traits are phenotypic characteristics that vary in degree and are typically attributed to the interactions between two or more genes and their environment. Quantitative trait loci (QTLs) are stretches of DNA that are closely linked to the genes that underlie the trait in question. A QTL may encompass 0, 1, or typically more than one gene. QTLs can be associated with any quantitative trait. In the most preferred embodiments, QTLs are associated with commercially valuable traits, for example antixenosis, anibiosis, tolerance or other traits that improve resistance to pests such as insects or increase crop yields.

Desirable QTL, transgenes, or combinations thereof, such as those disclosed herein, can be analyzed according to any method known in the art. For example, phenotypes can be assessed for improvement of one or more desirable traits using phenotype analysis techniques or genotyping.

For example, the presence of QTLs in a plant or plant cell can be associated or linked to regions of the genome that are contributing to variation in a trait of interest. Once the trait is associated or linked with one or more genetic markers, the genetic markers can be used to determine if a particular plant has the desirable QTL (i.e. a genetic region or chromosomal segment including the desirable QTL) or not.

Genomic regions can be analyzed using any method known in the art. For example, hybrid plants can be genotyped with restriction fragment length polymorphism (RFLP) markers. An RFLP is a difference in homologous DNA sequences that can be detected by the presence of fragments of different lengths after digestion of the DNA samples in question with specific restriction endonucleases. An RFLP probe is a labeled oligonucleotide sequence that hybridizes with one or more fragments of the digested DNA sample after they are separated by gel electrophoresis, thus revealing a unique blotting pattern characteristic to a specific genotype at a specific locus. Short, single- or low-copy genomic DNA or cDNA clones are typically used as RFLP probes.

Alternatively, QTLs can be identified by amplified fragment length polymorphism (AFLP). AFLP uses restriction enzymes to digest genomic DNA, followed by ligation of adaptors to the sticky ends of the restriction fragments. Restriction fragments are selected, and amplified by PCR using primers complementary to the adaptor sequence, the restriction site sequence and a few nucleotides inside the restriction site fragments. The amplified fragments are visualized (i.e. detection of a specific genotype at a specific locus) on denaturing polyacrylamide gels using, for example, autoradiography or fluorescence methodologies.

Other methods useful in QTL genotyping may include analysis of randomly amplified polymorphic DNA (RAPD), highly polymorphic short tandem repeat (STR) or simple sequence repeat (SSR) markers also referred to as microsatellites, or polymorphic single nucleotide polymorphisms (SNPs), or sequencing fragments of the genome (i.e., genomic sequencing). In some embodiments, the plants disclosed herein are genotyped according to random fragment length polymorphisms (RFLP) markers from a known genetic map. In this way, the presence of the desired QTL or QTLs can be monitored or tracked in the progeny of each successive round of a breeding program.

In some embodiments protoplast fusion is used transfer of nucleic acids from a donor plant to a recipient plant. Protoplast fusion is an induced or spontaneous union, such as a somatic hybridization, between two or more protoplasts (cells of which the cell walls are removed by enzymatic treatment) to produce a single bi- or multi-nucleate cell. The fused cell, that may even be obtained from plant species that cannot be interbred in nature, is tissue cultured into a hybrid plant exhibiting the desirable combination of traits. The protoplasts are then fused using traditional protoplast fusion procedures, which are known in the art.

In another embodiment, embryo rescue is employed to transfer a nucleic acid comprising one or more superior QTLs from a donor plant to a recipient plant. Embryo rescue can be used as a procedure to isolate embryo's from crosses wherein plants fail to produce viable seed. In this process, the fertilized ovary or immature seed of a plant is tissue cultured to create new plants.

### V. Screening Methods

Methods are also provided for identifying treatments, such as chemical treatments, that can modify expression or bioavailability of a flavonoid glucosyltransferase such as Glyma07g14530 in a plant.

In some embodiments, the method involves administering a candidate agent to a transgenic plant disclosed herein and comparing the effect of the administration on flavonoid glucosyltransferase activity in the plant to a control. For example, the purpose of the method can be to identify an agent that causes the transgenic plant to exhibit increased pest resistance.

In some embodiments, the method involves contacting cells expressing a flavonoid glucosyltransferase gene or an ortholog or homolog thereof with a candidate agent, monitoring the effect of the candidate agent on flavonoid glucosyltransferase gene expression, and comparing the effect of the candidate agent on flavonoid glucosyltransferase gene expression to a control. For example, the purpose of the method can be to identify an agent that promotes or reduces flavonoid glucosyltransferase gene expression. In these embodiments, a decrease in flavonoid glucosyltransferase gene expression would identify an agent that could be used to increase insect resistance. Likewise, the purpose of the method can be to identify an agent that increases flavonoid glucosyltransferase gene expression. In these embodiments, an increase in flavonoid glucosyltransferase gene expression would identify an agent that could be used to increase insect sensitivity.

Expression of a flavonoid glucosyltransferase, such as Glyma07g14530 gene expression can be detected using routine methods, such as immunodetection methods. The methods can be cell-based or cell-free assays. The steps of various useful immunodetection methods have been described in the scientific literature, such as, e.g., Maggio, et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In some embodiments, a reporter construct, such as a fluorochrome or enzyme, is operably linked to a Glyma07g14530 expression control sequence. In these embodiments, the purpose of the method can be to identify an agent that modulates activation of the Glyma07g14530 expression control sequence by detecting the effect of a candidate agent on reporter expression.

In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the disclosed screening procedure. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds.

Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

When a crude extract is found to have a desired activity, further fractionation of the positive lead can be used to isolate chemical constituents responsible for the observed effect. Thus, the goal of the extraction, fractionation, and purification process is the careful characterization and identification of a chemical entity within the crude extract having the activity. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases or conditions, such as those disclosed herein.

Candidate agents encompass numerous chemical classes, but are most often organic molecules, e.g., small organic compounds having a molecular weight of more than 100 and less than about 2,500 Daltons. Candidate agents comprise functional groups necessary for structural interaction with proteins, particularly hydrogen bonding, and typically include at least an amine, carbonyl, hydroxyl or carboxyl group, for example, at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. In a further embodiment, candidate agents are peptides.

### VI. Methods of Identifying Pest Resistance Genes in Related Plants

Methods are also provided for identifying genes that control insect resistance in other plants. Therefore, methods for identifying Glyma07g14530 homologs orthologs in plants are provided. The methods generally involve using the gene sequences for Glyma07g14530 disclosed herein.

In preferred embodiments, the plant is closely related to soybean. In some embodiments, the method involves scanning the genetic sequences of a plant for genes that are orthologous or homologous to Glyma07g14530.

In other embodiments, the method involves conducting a BLAST search of plant genomes for genes having the highest nucleic acid sequence identity to that of Glyma07g14530. For example, the orthologous gene can have 10%, 15%, 20%, 25%, 30%, 35%, 40%, 45%, 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the nucleic acid sequence SEQ ID NO: 1, 2, 3, 4, 5, or 6, or a nucleic acid encoding the amino acid sequence of SEQ ID NO:7, 8, or 9, or a fragment or variant thereof.

### VII. Methods of Genotyping Plants for Pest Resistance

A. Haplotypes

The sequences disclosed herein can be used to screen for pest resistance in plants. For example, the genotype of one or more insertions, deletions, and polymorphisms in or around Glyma07g14530 that reduce or inhibit expression of the Glyma07g14530 gene product, or lead to an altered gene product, such as a truncated protein or alternative splice variant, or a gene product with reduced function can be used to genotype a plant as insect resistant relative to a plant encoding a functional, full-length protein. For example, deletions, insertions, and polymorphisms can be determined by comparing the Glyma07g14530 sequence from a test plant to SEQ ID NO: 1, 2, or 3 using global sequence alignment tools.

A plant can also be determined to be pest resistant by detecting the non-synonymous SNP polymorphism G->A at nucleotide 837 of SEQ ID NO:4 causing a premature STOP codon of Glyma07g14530.

The process of determining which specific nucleotide (i.e., allele) is present at each of one or more SNP positions, such as a disclosed SNP position in the Glyma07g14530 gene locus, is referred to as SNP genotyping. Methods for SNP genotyping are generally known in the art (Chen et al., Pharmacogenomics J., 3(2):77-96 (2003)); Kwok, et al., Curr. Issues Mol. Biol., 5(2):43-60 (2003)); Shi, Am. J. Pharmacogenomics, 2(3):197-205 (2002)); and Kwok, Annu. Rev. Genomics Hum. Genet., 2:235-58 (2001)).

SNP genotyping can include the steps of collecting a biological sample from a plant, isolating genomic DNA from the cells of the sample, contacting the nucleic acids with one or more primers which specifically hybridize to a region of the isolated nucleic acid containing a target SNP under conditions such that hybridization and amplification of the target nucleic acid region occurs, and determining the nucleotide present at the SNP position of interest, or, in some assays, detecting the presence or absence of an amplification product (assays can be designed so that hybridization and/or amplification will only occur if a particular SNP allele is present or absent). In some assays, the size of the amplification product is detected and compared to the length of a control sample; for example, deletions and insertions can be detected by a change in size of the amplified product compared to a normal genotype.

The neighboring sequence can be used to design SNP detection reagents such as oligonucleotide probes and primers. In some embodiment probe or primers are designed based on the nucleic acid sequence disclosed herein, i.e., SEQ ID NO:1, 2, 3, 4, 5, or 6.

Common SNP genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, pyrosequencing, multiplex primer extension sorted on genetic arrays, ligation with rolling circle amplification, homogeneous ligation, multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection.

### EXAMPLES

### Example 1

### The Qualitative Trait Loci QTL-M Imparts Insect Resistance

In the late 1960s, Van Duyn, et al., Crop Science, 11:572-73 (1971) identified three soybean plant introductions (PI) from Japan with Host Plant Resistance (HPR) to chewing insects, namely PI171451, PI227687, and PI229358. These plants exhibited resistance to multiple insect pests, including a coleopteran such as the Mexican bean beetle (MBB), and several soybean lepidopteran pests such as soybean looper (SBL), velvetbean caterpillar (VBC), and beet armyworm (BAW) (Hatchett, et al., Crop Science, 16:277 (1976)); Lambert, et al., Crop Science, 24:887 (1984)).

Genetic mapping populations were created by crossing the insect-resistant genotypes PI229358 and PI171451 to the susceptible cultivar Cobb so that the insect-resistant loci could be identified using molecular markers (Rector et al., 1998; Rector et al., 2000; Rector et al., 2000)). A major QTL was identified on Linkage Group (LG) M (now chromosome 7) of PI229358 and PI171451, referred to as QTL-M. QTL-M is associated with both antixenosis and antibiosis resistance to CEW; accounting for 37% of the observed variance in antixenosis, and for 22 to 28% of the variance in antibiosis. This same region of chromosome 7 has been associated with resistance to the common cutworm in another Japanese soybean, PI594177 (Komatsu et al., 2010; Komatsu et al., 2008; Komatsu et al., 2005; Komatsu et al., 2004).

Besides QTL-M, other QTLs associated with resistance to CEW were also identified: (i) QTL-H (chromosome 12) confers antixenosis in PI229358 and PI171451, with R² values ranging from 9 to 19%; and (ii) QTL-G (chromosome 18), which confers antibiosis (R²=19%) in PI229358 (Rector et al., 2000)). However, QTLs G and H are only effective when QTL-M is present in the genome. In a set of eight advanced breeding lines, BC5F2-derived plants, it was determined that the main effect of QTL-H (antixenosis) and QTL-G (antibiosis) is not significant (P>0.05) when the allele at the QTL-M locus corresponds to the susceptible background; conversely, lines carrying the PI229358 allele for resistance at QTL-M were more resistant if the PI229358 alleles were present at QTL-H or QTL-G (Zhu et al., 2006)). By combining the earlier RFLP data with simple sequence repeat (SSR) markers, the locations of QTL-H, and QTL-M more precisely mapped in each chromosome (Narvel, et al., Crop Science, 41:1931 (2001), Zhu, et al., (2006)).

### Example 2

### QTL-M Enhances the Effectiveness of Bt in Soybean

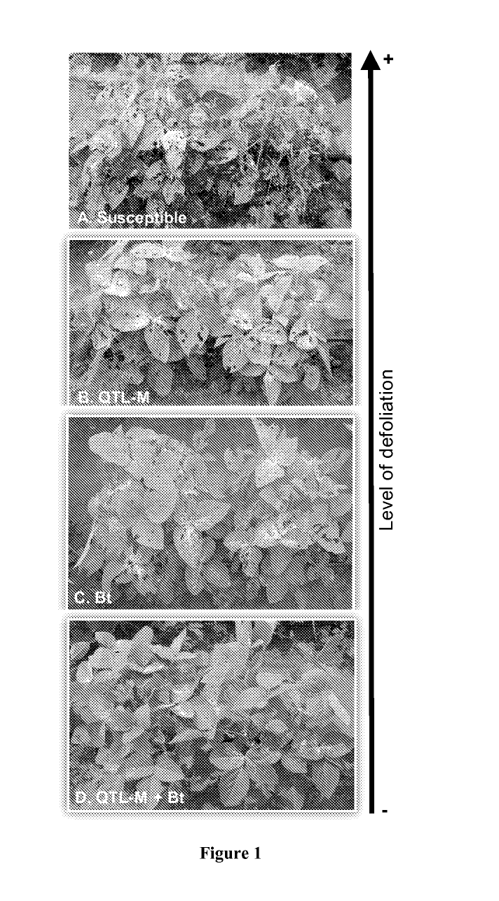

The effects of the resistant alleles for QTL-M and QTL-H from PI229358 on the effectiveness of Bt were also investigated. Jack^{Bt} (soybean cultivar Jack (Nickell, et al., Crop Science, 30:1365 (1990)) engineered with Bt) was crossed with PI229358, followed by two backcrosses to Jack^{Bt}. Jack^{Bt} expresses a synthetic Bacillus thuringiensis insecticidal crystal protein gene transgene (Stewart, et al., Plant Physiol, 112:121-29 (1996), and under field conditions (FIG. 1C) is resistant to CEW and VBC (Walker, et al., TAG Theoretical and Applied Genetics, 109:1051-57 (2004)). SSR markers were used for marker-assisted backcrossing to obtain individuals carrying the QTL-containing regions from PI229358. Then, F₃-derived lines with QTL-M and/or Cry1Ac were identified and evaluated for resistance (Walker, et al., Molecular Breeding, 9:43-51 (2002)). Antibiosis to CEW and SBL was measured as larval weight gain in bioassays with detached leaves sealed in Petri dishes. SBL larvae feeding on leaves from the line with both Bt and QTL-M had significantly lower weights than larvae feeding on plants with either resistance gene alone. Besides documenting that QTL-M enhances the effectiveness of Bt, the results are all the more relevant because SBL is not as sensitive to Cry1Ac as CEW and VBC (Walker, et al., Econ Entomol, 93:613-622 (2000)).

Other BC2F3 lines with Cry1Ac, QTL-H, and QTL-M in all possible combinations were developed in a similar fashion from the same cross, and were tested for two years in the field for resistance to CEW and SBL. These field trials confirmed the ability of QTL-M to enhance the effectiveness of Bt (FIG. 1D). In addition, the plants were tested with the YHD2 strain of tobacco budworm (TBW), which was selected for its resistance to Cry1Ac (Gould, et al., Proc Natl Acad Sci USA, 89:7986-990 (1992)). As expected, YHD2 caterpillars were not affected by Bt soybean, but the same strain feeding on Bt soybean with QTL-M suffered detrimental effects; thus illustrating the importance of additional, unrelated genes to enhance the effectiveness of Bt in soybean (Walker, et al., TAG Theoretical and Applied Genetics, 109:1051-57 (2004)). Overall, pyramiding Bt with genes with other modes of action is considered an effective strategy for durable resistance to insects (Coombs, et al., Journal of the American Society for Horticultural Science, 127:62-68 (2002)); Gould, et al., Proc Natl Acad Sci USA, 89:7986-990 (1992)); Roush, Series B: Biological Sciences, 353:1777-786 (1998)).

A set of Recombinant Near-isogenic Inbred Lines (RNILs) was also developed in a ‘Benning’ (Boerma, et al., Crop Science, 37:1982 (1997)) genetic background. These contain all possible combinations of Bt, QTL-M, QTL-H, and QTL-G. Insect bioassays with these lines once again revealed that QTLs H and G are only effective against caterpillars in the presence of QTL-M (Zhu, et al., Theor Appl Genet, 116:455-463 (2008)). The reason why QTL-H and QTL-G only contribute to caterpillar resistance in the presence of QTL-M remains unknown, but serves to further illustrate the importance of QTL-M. These lines also confirmed that Bt is more effective in the presence of HPR QTLs.

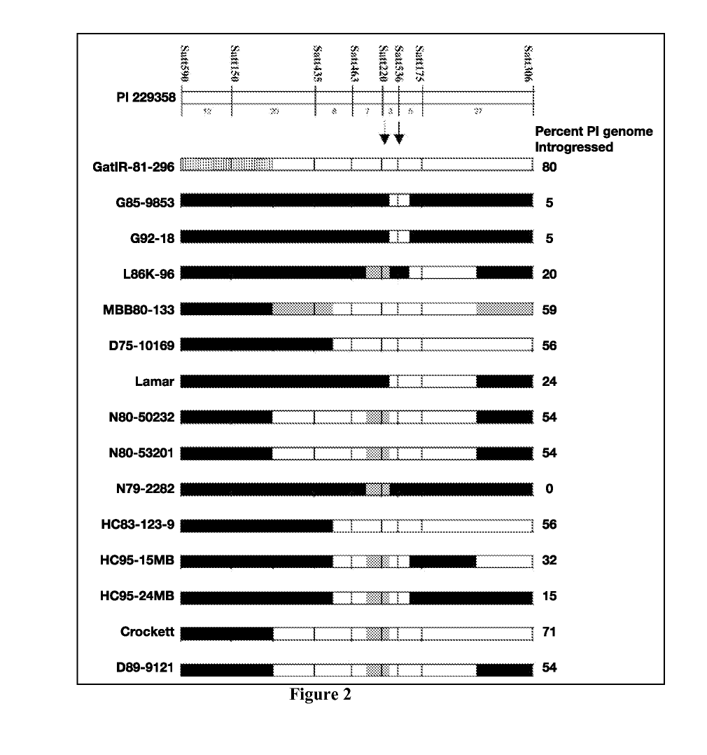

A retrospective genetic analysis of soybean genotypes bred for insect resistance further emphasized the importance of QTL-M and helped resolve its location in chromosome 7. SSRs were used to evaluate the success that breeders have had in introgressing insect resistance into soybean cultivars and breeding lines using only phenotypic selection (Narvel, et al., Crop Science, 41:1931 (2001)). Most of the 15 genotypes analyzed had PI229358 as the donor, though PI171451 was reported as the resistance donor for three lines/cultivars. Thirteen of the soybean genotypes examined had the PI229358 allele at QTL-M. In contrast, PI229358 alleles at QTL-G and QTL-H were only introgressed into two genotypes each. This marker data also provided strong evidence that QTL-M is in the 8-cM interval (FIG. 2) flanked by Satt220 and Satt175, and that Satt536 is the closest SSR marker to QTL-M (Narvel, et al., Crop Science, 41:1931 (2001)). It is noteworthy that none of these soybean genotypes have been agronomically successful due to negative traits associated with the donor parent DNA around QTL-M, or other PI-derived alleles (i.e., linkage drag).

These data illustrate the importance of QTL-M to control chewing insects because of its: (i) contribution to both antixenosis and antibiosis resistance, (ii) ability to enhance the effectiveness of Bt, (iii) detrimental effect on Bt-resistant insects, (iv) ability to ‘activate’ the effectiveness of QTLs G and H against caterpillars, and (v) broad range of effectiveness across multiple insect herbivore pests, including lepidopteran and at least one coleopteran species.

However, a gene conferring resistance to chewing insects has never been cloned in any plant, with the exception of genes for enzymes producing insecticidal compounds, such as maysin (Byrne, et al., Proceedings of the National Academy of Sciences, 93:8820 (1996)).

### Example 3

### QTL-M is in a 0.52-cM Segment of Chromosome 7

### Materials and Methods

Sequencing of Polymorphic Regions within QTL-M Polymorphic regions were detected in an alignment of the QTL-M region from PI229358 and Williams 82. Oligonucleotides flanking the polymorphisms were designed, and used to amplify genomic DNA from the panel of susceptible and resistant genotypes. To detect polymorphism unique to insect-resistant soybean genotypes, the PCR products were sequenced and assembled to the corresponding sequence of PI229358.

### Results

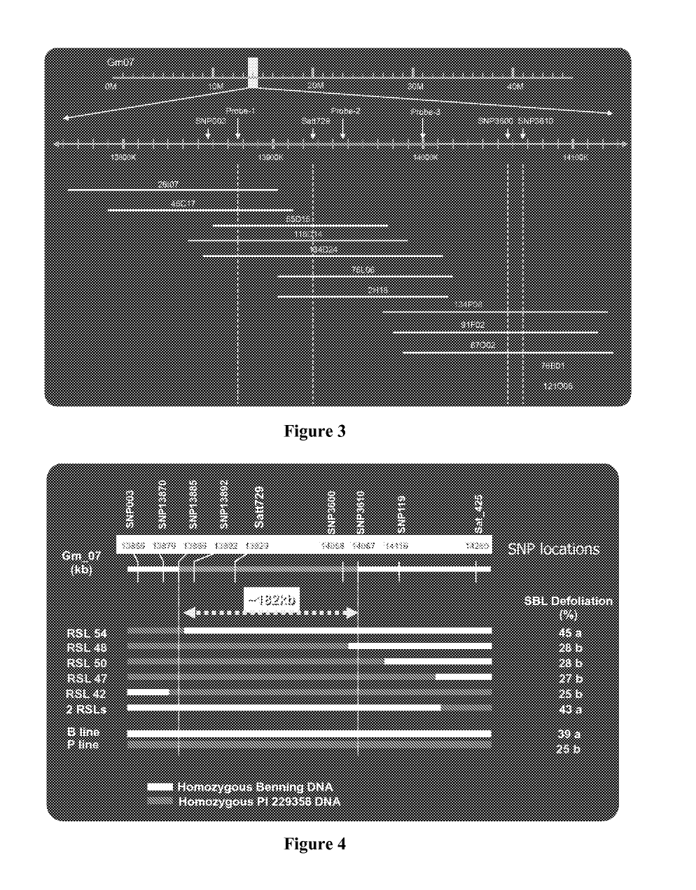

The location of QTL-M was narrowed down from an 8-cM to a 0.52-cM segment after developing and screening 1,911 recombinant substitution lines (RSLs). A BAC library was created from the insect-resistant PI229358, which is publicly available through the Clemson University Genomics Institute (Zhu, et al., Plant Molecular Biology Reporter, 27:229-235 (2009)). The SSR markers used to identify the RSLs containing QTL-M were used to find the location of this QTL in the draft genome of the cultivar Williams 82, which is susceptible to chewing insects. This sequence allowed the design of three DNA probes: two flanking, and one within QTL-M. Two overlapping BAC clones of the PI229358 library were identified using these probes (FIG. 3).

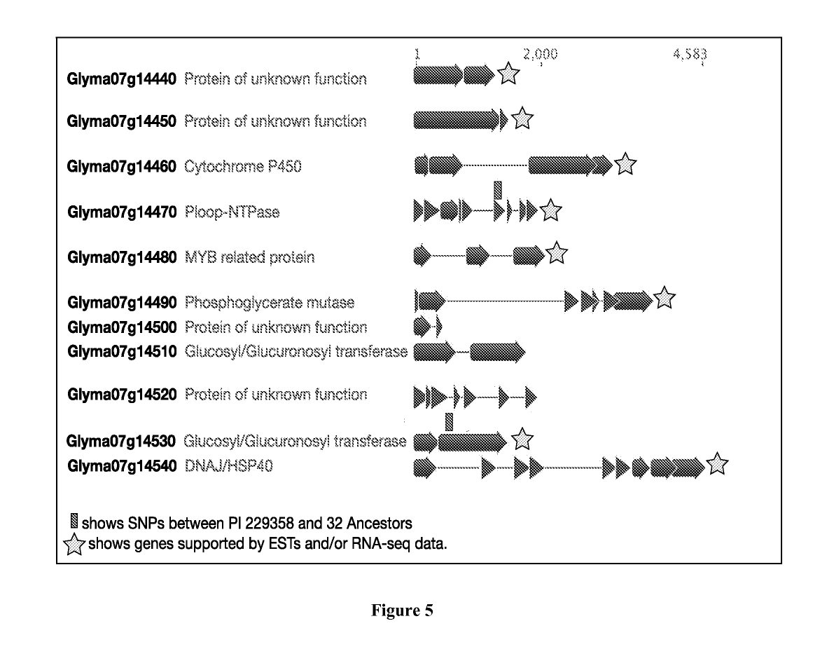

The sequence from the two BAC clones was obtained, namely 118D14 and 134P08, which are 150 kbp and 147 kbp, respectively. Their assembled sequence has 13 kbp overlap, and their contig corresponds to 284 kbp of the chromosome 7 from PI229358. The two SNP markers flanking QTL-M, SNP13885 and SNP3610, are contained within this sequence, 187 kbp apart from each other (FIG. 4). From the assembly of the BAC contig from PI229358 (insect resistant) to the genome of Williams 82 (insect susceptible) (Schmutz, et al., Nature, 463:178-183 (2010)), it was determined that QTL-M corresponds to 187 kb in PI229358, and 178 kbp in Williams 82. There are eleven annotated genes in the 178-kb sequence of Williams 82 (FIG. 5). Various mRNA transcripts (ESTs, RNA-seq) have been reported for eight of these genes; whereas the remaining three: Glyma07g14500, Glyma07g14510, and Glyma07g14520 correspond to low-confidence predicted hypothetical genes (Grant, et al., Nucleic Acids Res, 38:D843-46 (2010); Bolon, et al., BMC Plant Biol, 10:41 (2010); Severin, et al., BMC Plan Biol, 10:160 (2010)). The differences between the resistant PI229358 and susceptible Williams 82 sequences consist of: (i) 216 single nucleotide polymorphisms (SNPs), (ii) 68 indels, and (iii) a 5.4-kbp insertion in PI229358; and a 588 and a 599 bp deletion in PI229358 (Drummond, et al., http://www.geneious.com (2011)).

### Example 4

### Glyma07g14470 and Glyma07g14530 are QTL-M Candidate Genes

The large insertions/deletions were eliminated as candidates for conferring the QTL-M resistance, because they are not unique to the resistant genotypes. To identify SNPs associated with insect resistance, the coding and 5′-UTR regions of the eleven QTL-M genes, for a panel of 32 insect-susceptible genotypes that form most of the U.S. soybean ancestral germplasm pool (Gizlice, et al., Crop Science, 34:1143 (1994)), and 17 reported insect-resistant genotypes (Boethel, CRC Press, Boca Raton, Fla., (1999)) from Japan and China were sequenced. Two SNP loci were detected where alleles unique to insect-resistant soybeans were present; these SNPs were located in the genes Glyma07g14470, and Glyma07g14530.

Glyma07g14470 is a 1.9-kbp chimeric gene predicted to contain an SRP54-GTPase domain, a SNF2 N-terminal domain, a chloroplast target sequence, and a transmembrane domain. The SNP detected in Glyma07g14470 gene predicts the change in the proline (susceptible genotypes) residue at position 211 to leucine (8 resistant genotypes).

Glyma07g14530 is a putative flavonoid 3-O-glycosyltransferase, with 8 SNPs and 2 indels in the promoter region; and 3 SNPs in the coding sequence. One of the SNPs in the coding sequence is an allele present in 8 resistant genotypes, and absent from all of the 32 susceptible genotypes. In one embodiment, the SNP causes a premature stop codon, thus producing a truncated protein in the resistant genotypes (SEQ ID NO:4). In another embodiment, the SNP encodes an alternative splice site that alters that the structure and function of the protein.

### Example 5

### Glyma07g14530 Expression is Increased in Response to Insect Chewing

### Materials and Methods

Gene Expression

Total RNA was isolated from non-infested and infested soybean leaves. These total RNA samples were diluted to the same concentration, and used for RT-PCR reactions. Then, cDNA from each sample was generated using an oligo-dT primer, and it was used as template to amplify each one of the eleven QTL-M genes. A metalloprotease gene (Libault, et al., The Plant Genome, 1:44 (2008)) was used as a control in the RT-PCR reactions. Full-length cDNA for Glyma07g14530 was amplified from the same samples, using the SMARTer RACE cDNA amplification kit (Clontech Laboratories) and gene-specific oligonucleotides. The genomic clones of Glyma07g14530 were amplified from Jack (susceptible) and PI229358 (resistant), using the oligonucleotides 530-OE-F (ATT AGG CGC GCC ATG GAA TCA GCG GCA AGA AAC A) and 530-OE-R (CAC GCC TAG GTC AGC AAG TAG GAC GCA AAG).

### Results

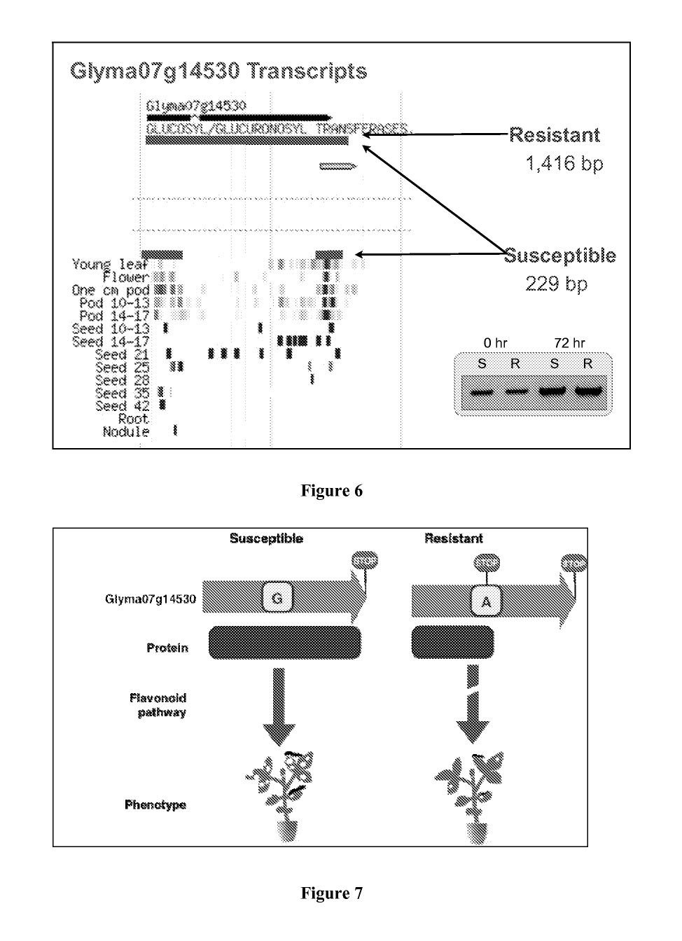

RT-PCR was conducted to confirm the expression of the genes within QTL-M; cDNA was obtained for the eight genes previously reported, and no transcripts were found for the low-confidence predicted hypothetical genes. RNA was also collected from leaves of 10-day-old plants of the susceptible cultivar, Benning, and its isoline BenningQ^{QTL-M}. After 72 hr of infestation with SBL, the RT-PCR reactions suggested an up-regulation of Glyma07g14530 in infested plants relative to non-infested ones (FIG. 6). In a subsequent experiment RNA was collected from Benning and BenningQ^{QTL-M} before infestation. A similar trend was observed for Glyma07g14530 at different time-points after infestation, with upregulation evident as early as 24 hr after infestation. In contrast, and based on the RT-PCR results, Glyma07g14470 appears to be a gene which transcription level is not particularly affected by herbivory.

### Example 6

### Glyma07g14530 Produces a Short and a Long Transcript in Leaves of Benning

### Materials and Methods

Full-Length cDNA Isolation and Candidate Gene Cloning To clone the full-length cDNA from Glyma07g14470, Arabidopsis thaliana, ecotype Landsberg-erecta, was transformed using the floral dip method (Clough, et al., Plant J, 16:735-743 (1998)) and the Agrobacterium tumefaciens strain GV3101 carrying a Glyma07g14470 expression construct. The full-length Glyma07g14470 cDNA isolated from Arabidopsis lacks the exon containing the SNP between susceptible and resistant soybean genotypes. The primers 470 Ara-cDNA-F (GTT GCA TGC TAG TTG CTG GGG ATG), and 470 Ara-cDNA-R (GCT GCT CCA CCT CTT GAA TCA CCA) were designed from this full-length cDNA; and these primers were used to amplify cDNA generated from soybean leaves. The RT-PCR product amplified from soybean leaves correspond to the same sequence amplified from Arabidopsis.

### Results

5′ and 3′ RACE-PCR reactions were performed to clone full-length cDNAs from Glyma07g14470 and Glyma07g14530. The full-length cDNA from Glyma07g14470 does not contain the predicted GTPase domain, which gets spliced out as an intron. As a result of the splicing, the non-synonymous SNP in resistant soybean does not have an effect on the Glyma07g14470 protein; therefore Glyma07g14530 is the gene conferring insect resistance in QTL-M.

Glyma07g14530 produces a short (229 bp) and a long (1,476 bp) transcript in leaves of Benning; only the long transcript is produced in leaves of BenningQ^{QTL-M} (FIG. 6). The long transcript indicates that Glyma07g14530 does not contain introns, as it is the same length as the genomic DNA. The lack of introns in glucosyltransferases genes that are associated with the modification of secondary metabolites is a common feature. As an example, 51 of the 88 glycosyltransferases predicted from Arabidopsis thaliana correspond to intronless genes (Li, et al., J Biol Chem, 276:4338-343 (2001)). Other biochemically characterized glucosyltransferases lacking introns have been reported (Kita, et al., FEBS Lett, 469:173-78 (2000); Ford, et al., J Biol Chem, 273:9224-233 (1998)).

### Example 7

### Sharp Trichomes Increase the Insect Resistance of QTL-M Greenhouse Tests

Antixenosis (choice test) was assayed in the greenhouse (All, Boerma and Todd 1989); in this study, the plants were tested for their resistance to defoliation by caterpillars. Each experiment will consist of a randomized complete block design with 15 replications: (i) plants from the different lines were grown in polystyrene cups that will be randomized within each replication; (ii) The blocks are arranged, as a plot, within a stainless steel pan; (iii) once the plants were 10 days old, the pans were filled with 2 cm of water; which provides irrigation to the plants, while containing any larvae which may fall from the plants; (iv) Each 10-day old plant was infested with four neonate larvae (CEW or SBL); (v) Larvae were allowed to feed on the plants for 10-14 days; during this period, once the foliage of neighboring plants comes into contact with each other, the larvae were able to move from plant to plant through the plot searching for a suitable substrate; (vi) At this point, the percentage of defoliation of each plant was estimated by at least three researchers; and the mean of the estimates for each plant was be used for an analysis of variance.

Detached Leaf Tests

Antibiosis (non-choice test) was assayed in a growth chamber, maintained at 27° C., 85% ambient humidity, and 14-hr photoperiod (provided by incandescent and fluorescent lights) (Walker, et al., Molecular Breeding (9):43-51 (2002); Zhu et al., Crop Science 46 (3): 1094 (2006)). In this case, the plants were be tested for their ability to reduce weight gain by chewing-insect larvae feeding on resistant leaves, relative to the weight gain of larvae feeding on leaves from susceptible plants. Each experiment consisted of a randomized complete block design with 15 replications: (i) Newly expanded trifoliolate leaves were collected from greenhouse-grown plants at the V4 (Fehr and Caviness 1977) stage; (ii) Each trifoliolate leaf was placed into a Petri dish containing plaster of Paris, which is saturated with water to keep enough moisture in each dish; (iii) Neonate larvae were placed within each Petri dish; three larvae per dish for SBL, and one larva per dish for CEW (CEW is cannibalistic if more than one larva is present); (iv) Each Petri dish was sealed, then placed in the growth chamber within the growth chamber each replication is arranged as a stack, with Petri dishes randomized within each stack; (v) On the 4^{th} day, a fresh trifoliolate leaf is added to each Petri dish; (vi) One week after starting the experiment, the Petri dishes are incubated for 24 hr at 4° C. to immobilize the larvae; (vii) Larvae from the each Petri dish were then transferred into a well of a microtiter plate; (viii) These plates were sealed, then frozen overnight at −20° C.; (ix) The larvae from each well get weighted as a group, and their mean weight is used for analysis of variance.

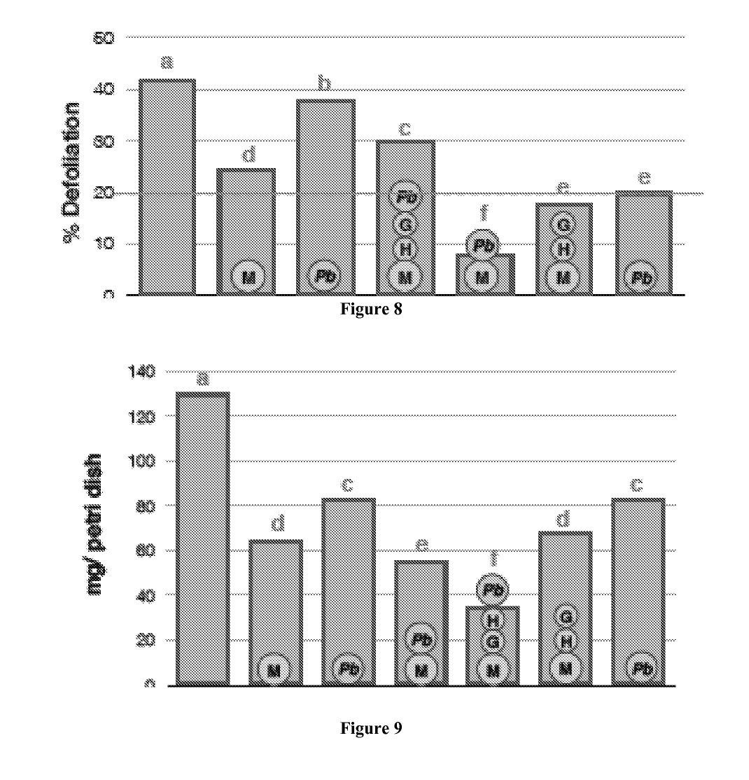

Near-isogenic lines of the cultivar Benning containing Pb and the QTLs in all possible combinations were developed through marker assisted selection. The isolines Benning^{QTL-M,Pb} and Benning^{QTL-M,G,Pb} were evaluated for their resistance to soybean looper in antixenosis (FIG. 8) and antibiosis bioassays (FIG. 9). The experiments consisted of fifteen completely randomized blocks.

### Field-Cage Test

The Benning isolines were grown in a randomized complete block design with fifteen replications at the Plant Sciences Farm. Hill plots were planted 30″ apart. Plots were over-seeded and later thinned to six plants per hill. The entire Each replication contained a hill of six plants from each Benning isoline. A nylon, fine-meshed cage was constructed over the experiment to create conditions for an artificial insect infestation (Rowan et al. 1991). Soybean looper neonate larvae were infested directly onto the plants at the V3 stage of development (Fehr and Caviness 1977). The larvae were applied at a rate of 80 per hill for 3 consecutive weeks. The cage prevented the invasion of predatory insects. The larvae were free to feed on the plants where they hatched, or migrate to adjacent plants. Visual defoliation ratings for the entire experiment were taken at 5, 7, 11, and 14 days after the first infestation. The percentage of defoliation was visually estimated for each hill. The defoliation data were checked for normality, and subjected to ANOVA.

Antixenosis Assay

In this assay, sharp trichomes significantly enhanced the effect of QTL-M in Benning^{QTL-M,Pb} plants, which exhibited 8.5% defoliation as compared to Benning^{QTL-M} (24%), and Benning (46%) (FIG. 8).

Antibiosis Assay

In this assay, 7-day old larvae feeding on Benning QTL-M,G,H,Pb weighed an average of 7 mg, whereas larvae feeding on Benning weighed 26 mg. The results indicate that pyramiding the QTLs from PI229358 with the sharp trichome gene from PI227687 is a good strategy to obtain agriculturally relevant levels of resistance (FIG. 9).

Field Cage Assay

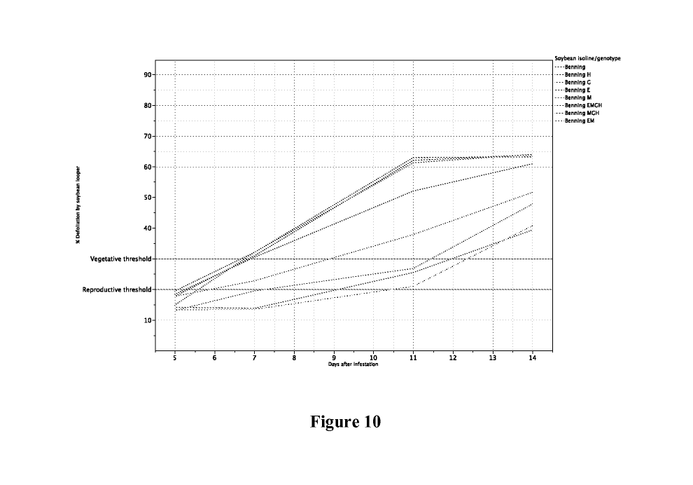

In this assay, sharp trichomes significantly enhanced the effect of QTL-M in Benning^{QTL-M,Pb} plants. This isoline exhibited the least defoliation (30%), respect to 63% for the susceptible Benning There was no significant difference between Benning^{QTL-M,Pb,} Benning^{QTL-M,G,H,} and Benning^{QTL-M,G,H,Pb} (FIG. 10). The results demonstrate that only two QTLs are required to achieve an economically significant level of resistance to leaf-chewing insects.

### Example 8

### QTL H Reduces Kudzu Bug Feeding

### Materials and Methods

Adult and larval feeding on wildtype (“Benning”) and various QTL hybrid soybean plants (“QTL M”, “QTL H”, and “QTL M/H”) were assayed: 5 replications in randomized complete block design with 2 rows per genotype (QTL hybrid). The total numbers of nymphs or adults were counted on 10 plants/row. The average number of bugs per plant was determined and the data was transformed using log (x+1).

A field experiment with a high level of kudzu bug infestation was harvested to compare yield between Benning (control) and QTL hybrids with QTL H. Soybean yield was obtained by harvesting two rows per genotype (QTL hybrid) for 5 replications. The beans collected were weighed and the data reported is in bushels (Bu); one Bu is equivalent to 60 lbs.

### Results

The data presented in FIG. 10 shows that the average number of kudzu bug larvae are reduced with both QTL M and QTL H, compared with Benning (control), or hybrids with only QTL M or only QTL H. These data indicate that the combination of QTLs M & H can control kudzu bug larvae.

The data presented in FIG. 11 shows that the average number of adult kudzu bugs is reduced on plants having QTL H compared to Benning (control). These data indicate that the presence of QTL H deters feeding by adult kudzu bugs.

The data presented in FIG. 12 shows that soybean yield is higher for the QTL hybrids that contain QTL H than for Benning (control), or other hybrids lacking QTL H. Fields under this level of kudzu bug infestation yield 20% lower than fields where the farmer sprays insecticides weekly to prevent infestations. The data show that soybean plants with QTL H yield 20% higher than for Benning (control).

1. The plant, or plant cell thereof of claim 40, wherein the plant is a transgenic plant comprising a polynucleotide comprising an expression control sequence operably linked to a nucleic acid sequence encoding an antisense nucleic acid that reduces, inhibits or silences expression or translation of a target polynucleotide comprising the nucleic acid sequence of SEQ ID NO: 1, 2, 3, or a complement thereof, or a nucleic acid sequence encoding the polypeptide of SEQ ID NO:7 or 8, or complement thereof.

2. The plant, or plant cell thereof of claim 1, wherein the antisense nucleic acid alters, reduces, inhibits, or silences expression or translation of the target polynucleotide by RNAi, dsRNA, miRNA, siRNA, or transacting small-interfering RNAs (tasiRNA).

3. A transgenic plant or transgenic plant cell comprising a recombinant polynucleotide comprising an expression control sequence of SEQ ID NO:10, or a fragment thereof comprising 50, 100, 150, 250, 500, 750, 1,000, 1,250, 1,500, or 2,000 or more nucleotides of SEQ ID NO:10 operably linked to a nucleic acid sequence encoding an insect resistance gene.

4. (canceled)

5. The transgenic plant of claim 3, wherein the insect resistance gene encodes a Bacillus thuringiensis protein, a derivative thereof, or a synthetic polypeptide modeled thereon.

6. The transgenic plant of claim 5, wherein the Bacillus thuringiensis protein is CryIAc, a derivative thereof, or a synthetic polypeptide modeled thereon.

7. (canceled)

8. The transgenic plant of claim 3, wherein the insect resistance gene encodes a polypeptide or protein selected from the group consisting of a lectin, an avidin, an enzyme inhibitor, an amylase inhibitor, a hormone, a pheromone, an antibody, and an immunotoxin.

9. (canceled)

10. (canceled)

11. The plant, or plant cell thereof of claim 42 further comprising an insect resistance QTL, wherein the insect resistance QTL is not QTL-M.

12. The plant, or plant cell thereof of claim 11, wherein the QTL is selected from the group consisting of Pb, QTL-H, QTL-G, and combinations thereof.

13. The plant, or plant cell thereof of claim 40, wherein the transgenic plant is a soybean plant.

14. The plant, or plant cell thereof of claim 13, wherein the soybean plant is selected from the group consisting of Jack, Resnik, Williams 82, Corsoy, Crawford, Hutcheson, Benning, Woodruff, Kunitz, and Champ.

15. The transgenic plant of claim 3, wherein the transgenic plant is a cereal crop a forage, a legume, a vegetable, or a specialty crop.

18. (canceled)

19. A seed from the plant according to claim 1.

20. A foodstuff comprising a plant part from the transgenic according to claim 1.

21. A method for increasing pest resistance in a plant comprising altering or reducing expression of Glyma07g14530 flavonoid glucosyltransferase or an ortholog thereof by an amount effective to increase pest resistance in the plant.

22. The method of claim 21, wherein expression of the Glyma07g14530 flavonoid glucosyltransferase is altered or reduced by reducing expression or translation of a target polynucleotide having a nucleic acid sequence according to SEQ ID NO: 1, 2, 3, or a complement thereof, or a nucleic acid encoding the polypeptide of SEQ ID NO:7 or 8, or a complement thereof.

23.-24. (canceled)

25. The method of claim 21, wherein the plant comprises at least one allele of Pb, QTL-G, QTL-H, or a combination thereof.

26.-37. (canceled)

38. A seed from the transgenic plant according to claim 3.

39. A foodstuff comprising a plant part from the transgenic plant according to claim 3.

40. A plant, or a plant cell thereof, genetically engineered to reduce or alter expression of Glyma07g14530 flavonoid glucosyltransferase or ortholog thereof relative the plant from which it is derived, wherein the plant exhibits increased pest resistance relative to the plant from which it is derived.

41. The plant, or plant cell thereof, of claim 40, wherein the reduced or altered expression comprising silencing of the Glyma07g14530 flavonoid glucosyltransferase or ortholog thereof, expression of a truncated or alternative splice variant of Glyma07g14530 flavonoid glucosyltransferase or ortholog thereof, or a combination thereof.

42. The plant, or plant cell thereof, of claim 40, wherein the plant does not comprise QTL-M.

43. A transgenic plant, or a plant cell thereof, expressing a recombinant truncated Glyma07g14530 flavonoid glucosyltransferase protein or ortholog thereof.

44. The transgenic plant of claim 43, wherein the transgenic plant is not a soybean plant.
